(19) United States
(12) Patent Application Publication
(10) Pub. No.: US 2026/0263358 A1
(43) Pub. Date: Sep. 10, 2026

(54) SILICONE PREPOLYMER AND CONTACT LENS FORMED THEREFROM

(71) Applicant: Bausch + Lomb Ireland Limited, Dublin (IE)

(72) Inventors: Alok Kumar Awasthi, Pittsford, NY (US); Sankarsan Biswas, Rochester, NY (US); Jade Russell, Perry, NY (US); Feng-Yang Shih, Lafayette, CO (US); James Anthony DiBella, JR., Penfield, NY (US); Anna Flach, Lafayette, CO (US); Vijay Singh, Webster, NY (US)

(21) Appl. No.: 19/544,512

(22) Filed: Feb. 19, 2026

Related U.S. Application Data
(60) Provisional application No. 63/766,738, filed on Mar. 4, 2025.

(51) Int. Cl. A61K 9/00; A61K 31/4725; B29D 11/00; B29K 83/00; B29L 11/00; C08F 2/50; C08F 290/06; G02B 1/04; G02C 7/04

(52) U.S. Cl. CPC A61K 9/0051; A61K 31/4725; B29D 11/00067; B29D 11/00134; C08F 2/50; C08F 290/068; G02B 1/043; G02C 7/04; B29K 2083/00; B29K 2995/002; B29L 2011/0041

(57) ABSTRACT

A silicone prepolymer includes a reaction product of (a) a copolymerization product of a polymerization composition containing (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group and (v) one of a lifitegrast or a salt thereof-containing monomer having an ethylenically unsaturated reactive group and a releasable linking group or a lifitegrast or a salt thereof-containing polymer having an ethylenically unsaturated reactive group and a releasable linking group, with (b) a monomer having a reactive functionality complementary to the hydroxyl moiety of the hydroxyalkyl acrylate monomer and a polymerizable ethylenically unsaturated reactive group.

### PRIORITY CLAIM

The present application claims priority to U.S. Provisional Patent Application Ser. No. 63/766,738, entitled “Silicone Prepolymer and Contact Lens Formed Therefrom,” filed Mar. 4, 2025, the content of which is incorporated by reference herein in its entirety.

### BACKGROUND

Contact lenses made from, for example, silicone-containing materials, have been investigated for a number of years. Such materials can generally be subdivided into two major classes, namely hydrogels and non-hydrogels. Hydrogels can absorb and retain water in an equilibrium state, whereas non-hydrogels do not absorb appreciable amounts of water. Regardless of their water content, both hydrogel and non-hydrogel silicone medical devices tend to have relatively hydrophobic, non-wettable surfaces that have a high affinity for lipids. This problem is of particular concern with contact lenses.

### SUMMARY

In accordance with an aspect of the present disclosure, a silicone prepolymer comprises a reaction product of (a) a copolymerization product of a polymerization composition comprising (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group, and (v) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (b) a monomer having a reactive functionality complementary to the hydroxyl moiety of the hydroxyalkyl acrylate monomer and a polymerizable ethylenically unsaturated reactive group.

In accordance with another aspect of the present disclosure, a silicone contact lens comprises a polymerization product of a silicone contact lens-forming mixture comprising a silicone prepolymer comprising a reaction product of (a) a copolymerization product of a polymerization composition comprising (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group, and (v) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (b) a monomer having a reactive functionality complementary to the hydroxyl moiety of the hydroxyalkyl acrylate monomer and a polymerizable ethylenically unsaturated reactive group.

In accordance with yet another aspect of the present disclosure, a method for making a silicone contact lens comprises:
(a) subjecting a silicone contact lens-forming mixture comprising a silicone prepolymer comprising a reaction product of (i) a copolymerization product of a polymerization composition comprising (1) an alkylacrylamide monomer, (2) a hydroxyalkyl acrylate monomer, (3) a non-bulky organosilicon-containing monomer having an ethylenically unsaturated reactive group, (4) a bulky siloxane monomer having an ethylenically unsaturated reactive group, and (5) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (ii) a monomer having a reactive functionality complementary to the hydroxyl moiety of the hydroxyalkyl acrylate monomer and a polymerizable ethylenically unsaturated reactive group to polymerization conditions to provide a polymerized silicone contact lens, and
(b) hydrating the polymerized silicone contact lens.

In accordance with still yet another illustrative embodiment, a method for delivering lifitegrast or salt thereof to an eye of a subject comprises:
(a) placing a silicone contact lens in an eye of a subject, wherein the silicone contact lens is a polymerization product of a silicone contact lens-forming mixture comprising a silicone prepolymer comprising a reaction product of (i) a copolymerization product of a polymerization composition comprising (1) an alkylacrylamide monomer, (2) a hydroxyalkyl acrylate monomer, (3) a non-bulky organosilicon-containing monomer having an ethylenically unsaturated reactive group, (4) a bulky siloxane monomer having an ethylenically unsaturated reactive group, and (5) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (ii) a monomer having a reactive functionality complementary to the hydroxyl moiety of the hydroxyalkyl acrylate monomer and a polymerizable ethylenically unsaturated reactive group to polymerization conditions to provide a polymerized silicone contact lens, and
(b) delivering the lifitegrast or salt thereof to the eye of the subject by contacting the silicone contact lens with one of light having a wavelength of about 300 nanometers (nm) to about 700 nm or with an esterases or enzymes on the eye, in the tear film or on the flora of the eye.

### DETAILED DESCRIPTION

Various illustrative embodiments described herein include a silicone prepolymer containing one of a lifitegrast or a salt thereof-containing monomer or polymer comprising an ethylenically unsaturated reactive group and a releasable linking group and silicone contact lenses made therefrom for delivering lifitegrast or a salt thereof to an eye of a subject for treatment of dry eye.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

A “prepolymer” refers to a (i) homopolymer containing a polymerizable functional group which can be functionalized (e.g., crosslinked and/or polymerized and or embedded in the polymer matrix) actinically or thermally or chemically to obtain a crosslinked and/or polymerized polymer having a molecular weight higher than the molecular weight of the starting polymer or a (ii) starting polymer prepared by crosslinking at least two monomers which can be functionalized (e.g., crosslinked and/or polymerized and or embedded in the polymer matrix) actinically or thermally or chemically to obtain a crosslinked and/or polymerized polymer having a molecular weight higher than the molecular weight of the starting polymer.

A “crosslinkable prepolymer” refers to a starting polymer which can be crosslinked to obtain a crosslinked polymer having a molecular weight higher than the molecular weight of the starting polymer.

Representative examples of ethylenically unsaturated reactive groups for use herein include, by way of example, a (meth)acrylate-containing reactive end group, a (meth)acrylamide-containing reactive end group, an allyl-containing reactive end group, a vinyl-containing reactive end group, a vinylcarbonate-containing reactive end group, a vinylcarbamate-containing reactive end group, a styrene-containing reactive end group, an itaconate-containing reactive end group, a vinyloxy-containing reactive end group, a fumarate-containing reactive end group, a maleimide-containing reactive end group, a vinylsulfonyl reactive end group and the like.

In a non-limiting illustrative embodiment, an example of a (meth)acrylate-containing reactive end group can be represented by the structure:

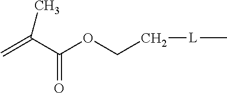

wherein L is a linking group or bond. Suitable linking groups include, for example, a heteroatom such as O, any divalent hydrocarbon radical or moiety such as independently a straight or branched, substituted or unsubstituted C₁-C₆ alkyl group, a substituted or unsubstituted C₃-C₁₂ cycloalkyl group, a substituted or unsubstituted C₄-C₁₂ cycloalkylalkyl group, a substituted or unsubstituted C₃-C₁₂ cycloalkenyl group, a substituted or unsubstituted C₆-C₁₂ aryl group, a substituted or unsubstituted C₇-C₁₂ arylalkyl group and substituted and unsubstituted ether-containing groups.

As used herein, the term “(meth)” denotes an optional methyl substituent. Thus, terms such as “(meth)acrylate” denotes either methacrylate or acrylate, and “(meth)acrylamide” denotes either methacrylamide or acrylamide.

As used in this disclosure, the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements with the exception of only minor traces of impurities.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

The weight average molecular weight of the silicone prepolymer is determined by Gel Permeation Chromatography (GPC).

As used herein, the term “contact lens” refers to contact lenses that reside in or on the eye. These lenses can provide optical correction, wound care, drug delivery, diagnostic functionality or cosmetic enhancement or effect or a combination of these properties. Suitable contact lenses include, for example, contact lenses such as soft contact lenses, e.g., a soft, hydrogel lens; soft, non-hydrogel lens and the like, hard contact lenses, e.g., a hard, gas permeable lens material, a hybrid lens and the like. A contact lens can be in a dry state or a wet state. A “dry state” refers to a soft contact lens in a state prior to hydration or the state of a hard lens under storage or use conditions. A “wet state” refers to a soft contact lens in a hydrated state. As is understood by one skilled in the art, a contact lens is considered to be “soft” if it can be folded back upon itself without breaking.

Ophthalmic drug delivery faces significant obstacles due to anatomical and physiological barriers, such as tear drainage and limited epithelial transport, which restrict drug bioavailability to around 5% or less. These barriers impede the penetration and absorption of therapeutic agents, making effective treatment challenging. An optimal delivery system must address these issues by enhancing bioavailability and enabling controlled drug release at the target site.

Lifitegrast or salts thereof are well-established medications for the treatment of dry eye disease. Ophthalmic lifitegrast is specifically used to alleviate the signs and symptoms associated with this condition. Classified as a lymphocyte function-associated antigen-1 (LFA-1) antagonist, lifitegrast or a salt thereof functions by reducing inflammation in the eye tissues.

Conventional methods like eye drops and ointments have been used to deliver lifitegrast or a salt thereof. However, these delivery methods are inefficient, requiring frequent applications and often resulting in poor patient compliance and incorrect dosing. While advanced formulations such as gels, viscous solutions, and colloidal systems have been developed, they have yet to achieve satisfactory outcomes.

The non-limiting illustrative embodiments disclosed herein overcome the foregoing drawbacks by providing a silicone prepolymer for making silicone contact lenses that can deliver lifitegrast or a salt thereof to the eye of the user in a continuous and sustained release manner. By providing significantly greater drug bioavailability to the cornea compared to the traditional methods discussed above, the silicone contact lenses of the non-limiting illustrative embodiments described herein can be utilized as a controlled drug delivery platform. This can be achieved by attaching one of a lifitegrast or a salt thereof-containing monomer or polymer comprising an ethylenically unsaturated reactive group and a releasable linking group to the silicone prepolymer backbone and forming a silicone contact lens from the silicone prepolymer. The lifitegrast or a salt thereof can then be delivered to an eye of a subject by contacting the contact lens with one of light having a wavelength of about 300 nanometers (nm) to about 700 nm or with an esterases or enzymes on the eye, in the tear film or on the flora of the eye thereby releasing the lifitegrast or a salt thereof in a continuous and sustained release.

A silicone prepolymer of the present disclosure comprises a reaction product of (a) a copolymerization product of a polymerization composition comprising (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group, and (v) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (b) a monomer having a reactive functionality complementary to the hydroxyl moiety of the hydroxyalkyl acrylate monomer and a polymerizable ethylenically unsaturated reactive group.

An alkylacrylamide monomer for use in the polymerization composition includes, for example, N,N-dimethylacrylamide, N,N-dimethylmethacrylamide, and the like.

The polymerization composition can contain from about 20 wt. % to about 60 wt. %, based on the total weight of the polymerization composition, of the alkylacrylamide monomer. In some embodiments, the polymerization composition can contain from about 40 wt. % to about 45 wt. %, based on the total weight of the polymerization composition, of the alkylacrylamide monomer.

A hydroxyalkyl acrylate monomer for use in the polymerization composition includes, for example, hydroxy C₁ to C₆ acrylate monomers. In some embodiments, a hydroxyalkyl acrylate monomer for use in the polymerization composition is represented by the following structure:

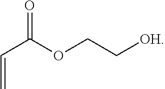

The polymerization composition can contain from about 0.01 wt. % to about 40 wt. %, based on the total weight of the polymerization composition, of the hydroxyalkyl acrylate monomer. In some embodiments, the polymerization composition can contain from about 20 wt. % to about 30 wt. %, based on the total weight of the polymerization composition, of the hydroxyalkyl acrylate monomer.

The polymerization composition further includes a non-bulky organosilicon-containing monomer having an ethylenically unsaturated reactive group. An “organosilicon-containing monomer” as used herein contains at least one [siloxanyl] or at least one [silyl-alkyl-siloxanyl] repeating unit, in a monomer, macromer or prepolymer. In an illustrative embodiment, an example of a non-bulky organosilicon-containing monomer is represented by a structure of Formula Ia:

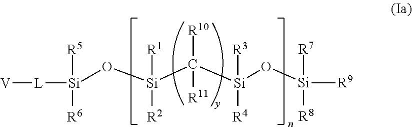

wherein V is an ethylenically unsaturated polymerizable group, L is a linking group or a bond; R¹, R², R³, R⁴, R⁵, R⁶, R⁷, R⁸, and R⁹ are independently hydrogen, an alkyl group, a haloalkyl group, a cycloalkyl group, a heterocycloalkyl group, an alkenyl group, a halo alkenyl group, or an aryl group; R¹⁰ and R¹¹ are independently hydrogen or an alkyl group wherein at least one of R¹⁰ and R¹¹ is hydrogen; y is 2 to 7 and n is 1 to 100 or from 1 to 20.

Ethylenically unsaturated polymerizable groups are well known to those skilled in the art. Suitable ethylenically unsaturated polymerizable groups include, for example, (meth)acrylates, vinyl carbonates, O-vinyl carbamates, N-vinyl carbamates, and (meth)acrylamides.

Linking groups can be any divalent radical or moiety and include, for example, a substituted or unsubstituted C₁ to C₁₂ alkyl group, an alkyl ether group, an alkenyl group, an alkenyl ether group, a halo alkyl group, a substituted or unsubstituted siloxane group, and monomers capable of propagating ring opening.

In one embodiment, V is a (meth)acrylate, L is a C₁ to C₁₂ alkylene group, R¹, R², R³, R⁴, R⁵, R⁶, R⁷, R⁸, and R⁹ are independently a C₁ to C₁₂ alkyl group, R¹⁰ and R¹¹ are independently H or a C₁ to C₁₂ alkyl group, y is 2 to 7 and n is 3 to 8.

In one embodiment, V is a (meth)acrylate, L is a C₁ to C₆ alkyl group, R¹, R², R³, R⁴, R⁵, R⁶, R⁷, R⁸, and R⁹ are independently a C₁ to C₆ alkyl group, R¹⁰ and R¹¹ are independently H or a C₁ to C₆ alkyl group, y is 2 to 7 and n is 1 to 20.

Non-bulky organosilicon-containing monomers represented by a structure of Formula Ia are known in the art, see, e.g., U.S. Pat. Nos. 7,915,323, 7,994,356, 8,420,711, 8,827,447 and 9,039,174, the contents of which are incorporated by reference herein.

In an illustrative embodiment, as may be combined with one or more of the preceding paragraphs, an example of a non-bulky organosilicon-containing monomer is represented by a structure of Formula Ib:

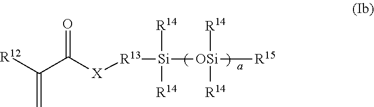

wherein R¹² is H or methyl; X is O or NR¹⁶, wherein R¹⁶ is selected from H, or C₁ to C₄ alkyl, which may be further substituted with one or more hydroxyl groups, and in some embodiments is H or methyl; R¹³ is a divalent alkyl group, which may further be functionalized with a group selected from the group consisting of ether groups, hydroxyl groups, carbamate groups and combinations thereof, and in another embodiment a C₁ to C₆ alkylene group which may be substituted with ether, hydroxyl and combinations thereof, and in yet another embodiment a C₁ or C₃ to C₄ alkylene group which may be substituted with ether, hydroxyl and combinations thereof; each R¹⁴ is independently a phenyl or a C₁ to C₄ alkyl group which may be substituted with fluorine, hydroxyl or ether, and in another embodiment each R¹⁴ is independently selected from ethyl and methyl groups, and in yet another embodiment, each R¹⁴ is methyl; R¹⁵ is a C₁ to C₄ alkyl group; a is 2 to 50, and in some embodiments 5 to 15.

Non-bulky organosilicon-containing monomers represented by a structure of Formula Ib are known in the art, see, e.g., U.S. Pat. Nos. 8,703,891, 8,937,110, 8,937,111, 9,156,934 and 9,244,197, the contents of which are incorporated by reference herein.

Representative examples of non-bulky organosilicon-containing monomers for use herein include:
M1EDS6-TMS: a compound having the structure and available from Gelest:

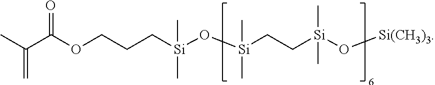

MCR-M11: a compound having the structure:

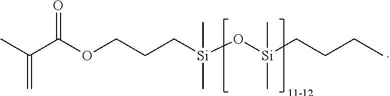

M1-MCR-C12: a compound having the structure:

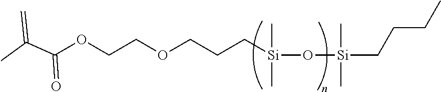

wherein n is an average of 12.

The polymerization composition can contain from about 0.10 wt. % to about 30 wt. %, based on the total weight of the polymerization composition, of a non-bulky organosilicon-containing monomer. In some embodiments, the polymerization composition can contain from about 5 wt. % to about 15 wt. %, based on the total weight of the polymerization composition, of a non-bulky organosilicon-containing monomer.

The polymerization composition further includes a bulky siloxane monomer having an ethylenically unsaturated reactive group. The term “bulky” refers to groups on the siloxane monomer that are sterically and/or electronically encumbering, i.e., sterically hindering. In a non-limiting illustrative embodiment, suitable bulky siloxane monomers include, for example, a bulky polysiloxanylalkyl (meth)acrylic monomer, a bulky polysiloxanylalkyl carbamate monomer and mixtures thereof. In one embodiment, a representative example of a bulky siloxane monomer is represented by a structure of Formula II:

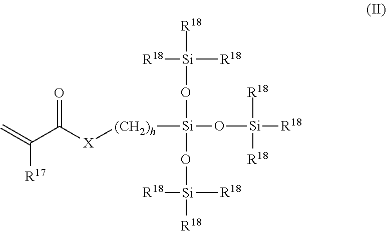

wherein X denotes —O— or —NR¹⁹—, where each R¹⁹ is hydrogen or a C₁-C₄ alkyl group; R¹⁷ independently denotes hydrogen or methyl; each R¹⁸ independently denotes a lower alkyl radical such as a C₁-C₆ group, a phenyl radical or a group represented by the following structure:

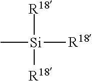

wherein each R^{18′} independently denotes a lower alkyl radical such as a C₁-C₆ group or a phenyl radical; and h is 1 to 10; or is represented by a structure of Formula III:

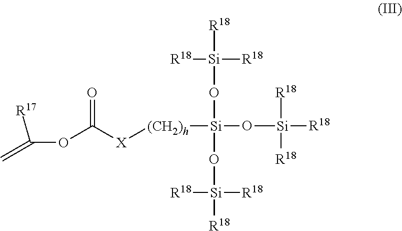

wherein X denotes —NR¹⁹— wherein R¹⁹ denotes hydrogen or a C₁-C₄ alkyl; R¹⁷ denotes hydrogen or methyl; each R¹⁸ independently denotes a lower alkyl radical such as a C₁-C₆ group, a phenyl radical or a group represented by the following structure:

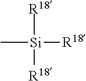

wherein each R^{18′} independently denotes a lower alkyl radical such as a C₁-C₆ group or a phenyl radical; and h is 1 to 10.

Representative examples of bulky siloxane monomers include 3-methacryloyloxypropyltris(trimethylsiloxy)silane or tris(trimethylsiloxy)silylpropyl methacrylate, sometimes referred to as TRIS and tris(trimethylsiloxy)silylpropyl vinyl carbamate, sometimes referred to as TRIS-VC, pentamethyldisiloxanyl methylmethacrylate, phenyltetramethyl-disiloxanylethyl acetate, and methyldi(trimethylsiloxy)methacryloxymethyl silane, (3-methacryloxy-2-hydroxy propoxy)propyl bis(trimethyl siloxy)methyl silane, sometimes referred to as Sigma and the like and mixtures thereof. In one embodiment, the bulky siloxane monomer is a tris(trialkylsiloxy)silylalkyl methacrylate-containing monomer such as a tris(trimethylsiloxy)silylpropyl methacrylate-containing monomer.

Such bulky monomers may be copolymerized with a silicone macromonomer, which is a poly(organosiloxane) capped with an unsaturated group at two or more ends of the molecule. U.S. Pat. No. 4,153,641 discloses, for example, various unsaturated groups such as acryloxy or methacryloxy groups.

The polymerization composition can contain from about 5 wt. % to about 30 wt. %, based on the total weight of the polymerization composition, of a bulky organosilicon-containing monomer. In some embodiments, the polymerization composition can contain from about 15 wt. % to about 25 wt. %, based on the total weight of the polymerization composition, of a bulky organosilicon-containing monomer.

The polymerization composition further includes one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group. In general, lifitegrast is represented by the following structure:

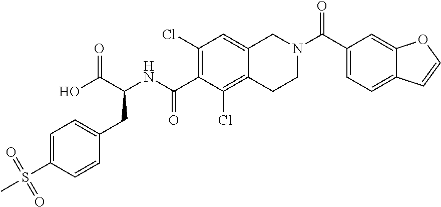 [SMILES: CS(=O)(=O)c1ccc(C[C@H](NC(=O)c2c(Cl)cc3c(c2Cl)CCN(C(=O)c2ccc4ccoc4c2)C3)C(=O)O)cc1]

and a salt of lifitegrast is represented by the following structure:

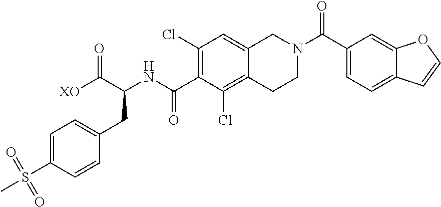

where x is a salt such as a sodium salt.

In some embodiments, the lifitegrast or salt thereof-containing monomer or lifitegrast or salt thereof-containing polymer may have one or more releasable linking groups. In some embodiments, a releasable linking group refers to a linking group that includes at least one bond that can be broken under physiological conditions, e.g., where the releasable linking group is a group linking the lifitegrast or salt thereof to a monomer or polymer comprising an ethylenically unsaturated reactive group via a pH-labile bond, an acid-labile bond, a base-labile bond, an oxidatively labile bond, a metabolically labile bond, a biochemically labile bond or an enzyme-labile bond, or other conditions such as a standard chemical reaction via a chemical-labile bond, e.g., a hydrolysis or substitution reaction, or via a photo-labile bond, e.g., exposure to light such as UV, near UV or visible light, e.g., a photosensitive bond. If the releasable linking groups are naturally cleaved under physiological conditions or cellular physiological conditions, then the monomer or polymer is biodegradable. In some embodiments, the releasable linking groups results in a loss of the lifitegrast or salt thereof from side chain atom of the lifitegrast or salt thereof-containing monomer or lifitegrast or salt thereof-containing polymer thereby releasing the lifitegrast or salt thereof.

In some embodiments, the releasable linking group includes ester groups. In the case of ester bonds, the esters undergo hydrolysis and are also catalytically cleaved by esterases thereby releasing the lifitegrast or salt thereof. Representative examples of a lifitegrast or a salt thereof-containing monomer having an ester linking group includes those having the following structures:

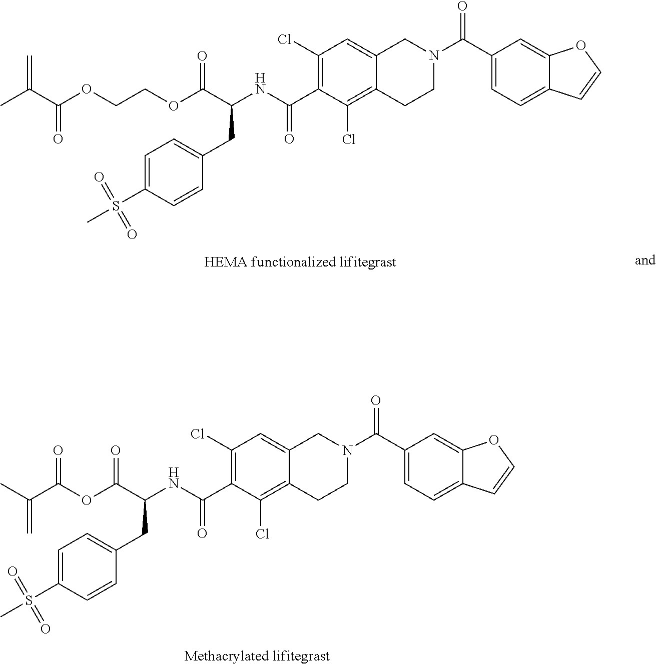 [SMILES: C=C(C)C(=O)OCCOC(=O)[C@H](Cc1ccc(S(C)(=O)=O)cc1)NC(=O)c1c(Cl)cc2c(c1Cl)CCN(C(=O)c1ccc3ccoc3c1)C2 ; C=C(C)C(=O)OC(=O)[C@H](Cc1ccc(S(C)(=O)=O)cc1)NC(=O)c1c(Cl)cc2c(c1Cl)CCN(C(=O)c1ccc3ccoc3c1)C2]

In some embodiments, the releasable linking group includes a photocleavable linking group. Suitable photocleavable linking groups include, for example, a nitrobenzyl group, phenacyl group, pyronin group, benzoin group and coumarin group. In one aspect, the photocleavable linking group has at least two reactive groups. The first reactive group allows linking of the photocleavable linking group to the ethylenically unsaturated reactive group. The second reactive group allows linking of the photocleavable linking group to the lifitegrast or salt thereof. The former handle is preferably stable, and the latter handle is preferably amenable to photolysis such that the lifitegrast or salt thereof is released from the photocleavable lifitegrast conjugate upon exposure to light of the appropriate wavelength. The photocleavable group therefore breaks its bond with the lifitegrast or salt thereof, removing the charged groups from the lifitegrast or salt thereof. The lifitegrast or salt thereof is then in its native form with no additions.

A representative example of a lifitegrast or a salt thereof-containing monomer having a photocleavable linking group is represented by the following structure:

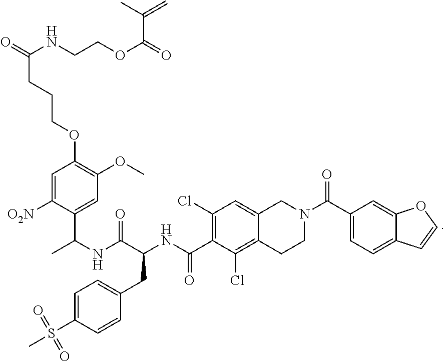 [SMILES: C=C(C)C(=O)OCCNC(=O)CCCOc1cc([N+](=O)[O-])c(C(C)NC(=O)[C@H](Cc2ccc(S(C)(=O)=O)cc2)NC(=O)c2c(Cl)cc3c(c2Cl)CCN(C(=O)c2ccc4ccoc4c2)C3)cc1OC]

In some embodiments, a lifitegrast or a salt thereof-containing polymer comprising an ethylenically unsaturated reactive group and a releasable linking group can include, for example, a lifitegrast or a salt thereof-containing glycosaminoglycan (GAG) polymer comprising a GAG having a polymer backbone and a first side chain comprising an ethylenically unsaturated reactive-containing residue grafted onto the polymer backbone and a second side chain comprising a lifitegrast or salt thereof residue grafted onto the polymer backbone. In other words, the ethylenically unsaturated reactive-containing residue grafted onto the GAG will have a reactive functional group in the polymer backbone capable of being grafted onto a reactive functional group of an ethylenically unsaturated reactive monomer and a reactive functional group in the polymer backbone capable of being grafted onto a reactive functional group of lifitegrast or salt thereof. A GAG is one molecule with many alternating subunits. In general, GAGs are represented by the formula A-B-A-B-A-B, where A is uronic acid and B is an amino sugar that may or may not be either O- or N-sulfated, where the A and B units can be heterogeneous with respect to epimeric content or sulfation. Any natural or synthetic polymer containing uronic acid can be used. Other GAGs are sulfated at different sugars. There are many different types of GAGs having commonly understood structures such as, for example, chondroitin sulfate (e.g., chondroitin 4- and 6-sulfates), heparan, heparin sulfate, heparosan, dermatan, dermatan sulfate, hyaluronic acid or a salt thereof, e.g., sodium hyaluronate or potassium hyaluronate, keratan sulfate, and other disaccharides such as sucrose, lactulose, lactose, maltose, trehalose, cellobiose, mannobiose and chitobiose. GAGs can be purchased from Sigma, and many other biochemical suppliers such as HTL Biotechnology (France). In an illustrative embodiment, the GAG is hyaluronic acid. In another illustrative embodiment, the GAG is chondroitin sulfate.

In an illustrative embodiment, a GAG for use herein can have a weight average molecular weight ranging from about 10,000 to about 3,000,000 Daltons (Da) in which the lower limit is from about 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about up to 2,800,000 Da, where any of the lower limits can be combined with any of the upper limits. In an illustrative embodiment, a GAG for use herein can have a weight average molecular weight ranging from about 1,000,000 to about 3,000,000 Da.

Hyaluronic acid is a well-known, naturally occurring, water soluble biodegradable polymer composed of two alternatively linked sugars, D-glucuronic acid and N-acetylglucosamine, linked via alternating β-(1,4) and β-(1,3) glycosidic bonds. Hyaluronic acid is a non-sulfated GAG. The polymer is hydrophilic and highly viscous in aqueous solution at relatively low solute concentrations. It often occurs naturally as the sodium salt, sodium hyaluronate. Methods of preparing commercially available hyaluronan and salts thereof are well known. Hyaluronan can be purchased from Seikagaku Company, Clear Solutions Biotech, Inc., Pharmacia Inc., Sigma Inc., HTL Biotechnology, Contipro and Bloomage Biotechnology Corporation, and many other suppliers. Hyaluronic acid has repeating units of the structure represented by the following formula:

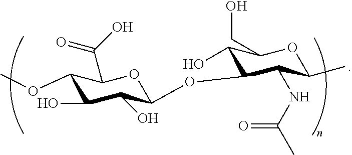

Accordingly, the repeating units in hyaluronic acid can be as follows:

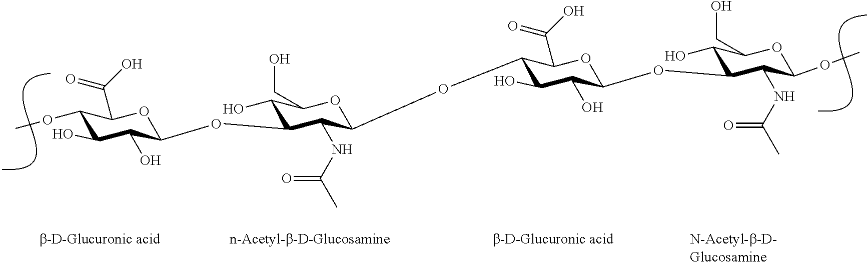

In general, hyaluronic acid or a salt thereof can have from about 2 to about 1,500,000 disaccharide units. In an embodiment, hyaluronic acid or a salt thereof can have a weight average molecular weight ranging from about 10,000 to about 3,000,000 Da in which the lower limit is from about 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about up to 2,800,000 Da, where any of the lower limits can be combined with any of the upper limits.

Chondroitin sulfate is a linear sulfated polysaccharide composed of repeating β-D-glucuronic acid (GlcA) and N-acetyl-β-D-galactosamine (GalNAc) units arranged in the sequence by GlcA-B (1,3)-GalNAc-B (1,4) glycosidic bonds. In an embodiment, chondroitin sulfate has one or more repeating units of the structure represented by the following formula:

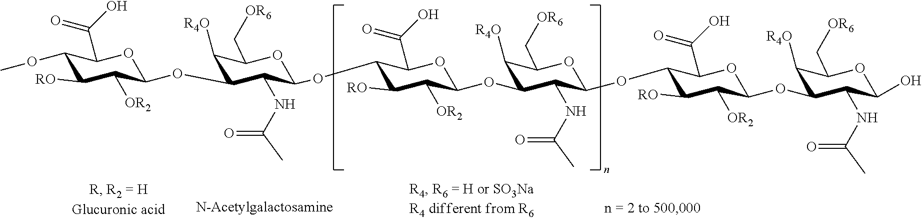

In an illustrative embodiment, chondroitin sulfate has repeating units of the structure represented by the following formula:

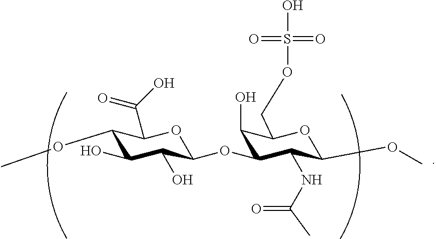

In general, chondroitin sulfate can have from about 2 to about 1,500,000 repeating units. In an embodiment, chondroitin sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about 3,000,000 Da where any of the lower limits can be combined with any of the upper limits or any of the upper limits can be combined with any of the upper limits.

In an illustrative embodiment, dermatan sulfate has repeating units of the structure represented by the following formula:

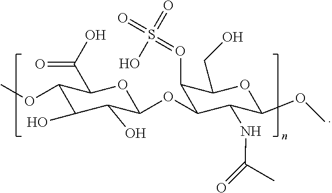

In general, dermatan sulfate can have from about 2 to about 1,500,000 repeating units. In an embodiment, dermatan sulfate can have a weight average molecular weight ranging from about 1,000 to about 2,000,000 Da in which the lower limit is from about 1,000, 5,000, 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about 2,000,000 Da where any of the lower limits can be combined with any of the upper limits or any of the upper limits can be combined with any of the upper limits.

In an illustrative embodiment, heparin and heparin sulfate has repeating units of the structure represented by the following formula:

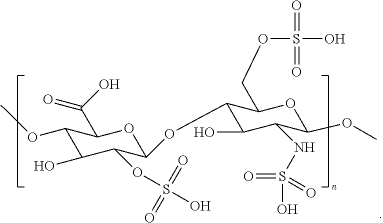

In general, heparin and heparin sulfate can have from about 2 to about 1,500,000 repeating units. In an embodiment, heparin and heparin sulfate can have a weight average molecular weight ranging from about 1,000 to about 3,000,000 Da in which the lower limit is from about 1,000, 5,000, 10,000, about 20,000, about 30,000, about 40,000, about 50,000, about 60,000, about 70,000, about 80,000, about 90,000, or about 100,000 Da, and the upper limit is about 40,000, 100,000, 200,000, about 300,000, about 400,000, about 500,000, about 600,000, about 700,000, about 800,000, about 900,000, about 1,000,000, or about 3,000,000 Da where any of the lower limits can be combined with any of the upper limits or any of the upper limits can be combined with any of the upper limits.

In an illustrative embodiment, keratan sulfate has repeating units of the structure represented by the following formula:

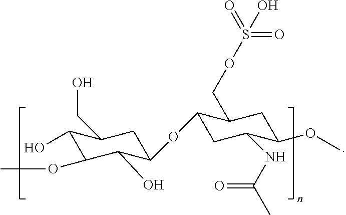

In general, keratan sulfate can have from about 2 to about 1,500,000 repeating units. In an embodiment, keratan sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000 Da, and the upper limit is about 100,000, 200,000, about 300,000, about 400,000, about 500,000, about 550,000, 600,000, about 700,000, about 800,000, about 900,000, about 1,000,000, or about 3,000,000 Da where any of the lower limits can be combined with any of the upper limits or any of the upper limits can be combined with any of the upper limits.

The ethylenically unsaturated reactive-containing residue grafted onto a reactive functional group in the polymer backbone of the GAG is derived from a monomer comprising an ethylenically unsaturated reactive group and at least one reactive end group. In one embodiment, the ethylenically unsaturated reactive-containing residue is a methacrylate-containing residue. The at least one reactive end group includes a reactive functional group capable of grafting on to a complementary reactive functional group in the polymer backbone of the GAG. Suitable reactive functional groups of the monomer comprising an ethylenically unsaturated reactive group and at least one reactive end group include, for example, a halogen, an anhydride, an amino group, an aldehyde group, a carboxylic acid group, an alcohol group, a thiol group, a hydrazide group, a glycidyl group, etc. In one non-limiting illustrative embodiment, an ethylenically unsaturated reactive-containing residue can be derived from, for example, methacrylic anhydride, methacryloyl chloride, 2-isocyanoethylmethacrylate, 3-(trimethoxysilyl)propyl methacrylate, 3-(chlorodimethylsilyl)propyl methacrylate, glycidyl methacrylate, methacryloyl hydrazide, aminoethyl methacrylate, vinyl chloroformate, allyl chloride, acryloyl chloride, and acrylic anhydride. However, other monomers for forming the ethylenically unsaturated reactive-containing residue on the polymer backbone of the GAG are contemplated and the foregoing list is merely exemplary.

The grafted lifitegrast or salt thereof-containing GAG polymers disclosed herein can be obtained by grafting the at least one reactive end group of the one or more monomers comprising an ethylenically unsaturated reactive group onto a complementary reactive functionality in the polymer backbone of the GAG and grafting having a reactive end group of the releasable linking group of the lifitegrast or salt thereof onto another complementary reactive functionality in the polymer backbone of the GAG. For example, in one illustrative embodiment, an anhydride group of the one or more monomers comprising an ethylenically unsaturated reactive group can be grafted onto a hydroxyl group in the polymer backbone of the GAG and the lifitegrast or salt thereof comprising a releasable linking group having a reactive end group can be attached onto another hydroxyl group in the polymer backbone of the GAG. In non-limiting illustrative embodiments, the graft polymerization reaction can obtain a degree of grafting, i.e., the number of sidechains in the polymer backbone containing the ethylenically unsaturated reactive-containing residue, ranging from about 0.5 to about 50%. In another illustrative embodiment, the degree of grafting can range from about 2 to about 30%. In another illustrative embodiment, the degree of grafting can range from about 5 to about 20%. In yet another illustrative embodiment, the degree of grafting can range from about 5 to about 15%. In yet another illustrative embodiment, the degree of grafting can range from about 5 to about 10%.

In general, the GAG, monomer comprising an ethylenically unsaturated reactive group and at least one reactive end group and the lifitegrast or salt thereof comprising a releasable linking group having a reactive end group can be added sequentially or simultaneously to a reaction mixture. The reaction can be carried out at a suitable temperature and for a time period for the completion of the reaction to maximize the yield of the product ethylenically unsaturated reactive-containing residue grafted onto the polymer backbone of the GAG. For example, a suitable temperature and time period includes a temperature ranging from about 10° C. to about 40° C. and a time period ranging from about 4 hours to about 48 hours. In an illustrative embodiment, a suitable temperature and time period includes a temperature ranging from about 15° C. to about 25° C. and a time period ranging from about 8 hours to about 24 hours.

The GAG can be added to the reaction mixture in an amount ranging from about 0.5 wt. % to about 5 wt. %, based on the total weight of the reaction mixture. In one illustrative embodiment, a GAG can be added to the reaction mixture in an amount ranging from about 1 wt. % to about 3 wt. %, based on the total weight of the reaction mixture.

In an illustrative embodiment, a monomer comprising an ethylenically unsaturated reactive group and at least one reactive end group can be added to the reaction mixture in an amount ranging from about 0.1 wt. % to about 5 wt. %, based on the total weight of the reaction mixture. In one illustrative embodiment, a monomer comprising an ethylenically unsaturated reactive group and at least one reactive end group can be added to the reaction mixture in an amount ranging from about 0.5 wt. % to about 2 wt. %, based on the total weight of the reaction mixture.

In a non-limiting illustrative embodiment, the ethylenically unsaturated reactive-containing residue is a methacrylate-containing residue derived from a methacrylate-containing monomer as described above, and the degree of methacrylation can range from about 0.5 to about 50%. In another illustrative embodiment, the degree of methacrylation can range from about 2 to about 30%. In yet another illustrative embodiment, the degree of methacrylation can range from about 5 to about 15%.

The lifitegrast or salt thereof-containing GAG polymer can be a random copolymer or a block copolymer. In one illustrative embodiment, a lifitegrast or a salt thereof-containing polymer disclosed herein can have a weight average molecular weight ranging from about 20,000 to about 6,000,000 Da in which the lower limit is from about 20,000, about 30,000, about 40,000, about 50,000, about 60,000, about 70,000, about 80,000, about 90,000, or about 100,000 Da, and the upper limit is about 100,000, about 150,000, about 200,000, about 300,000, about 400,000, about 500,000, about 600,000, about 700,000, about 800,000, about 900,000, about 1,000,000, about 2,000,000, about 3,000,000, about 4,000,000, about 5,000,000 or up to about 6,000,000 Da, wherein any of the lower limits can be combined with any of the upper limits.

A representative example of a lifitegrast or a salt thereof-containing GAG polymer is set forth below:

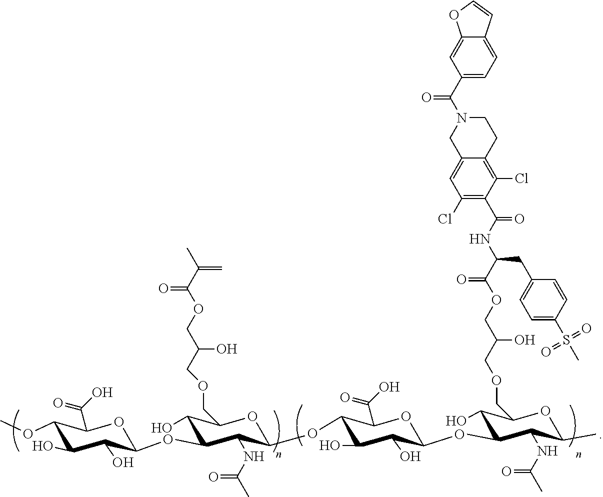

In non-limiting illustrative embodiments, as may be combined with one or more of the preceding paragraphs, the polymerization composition can further include an ultraviolet (UV) blocker having an ethylenically unsaturated reactive group. Suitable UV blockers having an ethylenically unsaturated reactive group can be any known UV blocker having an ethylenically unsaturated reactive group. In some embodiments, a UV blocker for use herein can include a phenolic group having a hydroxyl moiety and one or more ethylenically unsaturated reactive groups. In a non-limiting illustrative embodiment, a UV blocker comprising a phenolic group having a protected hydroxyl moiety and one or more ethylenically unsaturated reactive groups can be represented by a benzotriazole compound having a structure of Formula IV

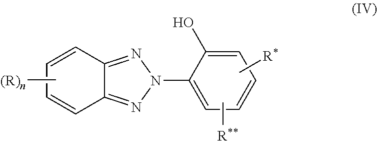

wherein each R is independently hydrogen, a halogen, an —O— group, a nitro group, a nitrile group, a substituted or unsubstituted alkoxy group, a substituted or unsubstituted hydroxyl group, a substituted or unsubstituted amine group, a substituted or unsubstituted carbonyl group, and a substituted or unsubstituted hydrocarbyl group, R* is hydrogen or a substituted or unsubstituted hydrocarbyl group, and R** is an ethylenically unsaturated reactive group.

As used herein, recitations of a “substituted” group means a group such as an alkyl group, cycloalkyl group, heterocycloalkyl group, aryl group, and/or heteroaryl group, in which at least one hydrogen atom thereof has been optionally replaced or substituted with a group that is other than hydrogen, such as, for example, halo groups (e.g., F, Cl, I, and Br), hydroxyl groups, ether groups, thiol groups, thio ether groups, carboxylic acid groups, carboxylic acid ester groups, phosphoric acid groups, phosphoric acid ester groups, sulfonic acid groups, sulfonic acid ester groups, nitro groups, cyano groups, hydrocarbyl groups (e.g., alkyl; alkenyl; alkynyl; cycloalkyl, including poly-fused-ring cycloalkyl and polycyclocalkyl; heterocycloalkyl; aryl, including hydroxyl substituted aryl, such as phenol, and including poly-fused-ring aryl; heteroaryl, including poly-fused-ring heteroaryl; and aralkyl groups), and amine groups.

As used herein, recitations of a “linear or branched” group, such as a linear or branched alkyl, are herein understood to include, for example, groups that are linear, such as linear C₂ to C₃₀ alkyl groups; and groups that are appropriately branched, such as branched C₃ to C₃₀ alkyl groups.

Representative examples of halogen groups include, by way of example, Cl, I, F, and Br.

Representative examples of hydrocarbyl groups include linear or branched alkyl groups, linear or branched alkenyl groups, linear or branched alkynyl groups, cycloalkyl groups, heterocycloalkyl groups, aryl groups (including polycyclic aryl groups), heteroaryl groups (having at least one hetero atom in the aromatic ring); and aralkyl groups as defined herein.

Representative examples of alkoxy groups for use herein include, by way of example, an alkyl group as defined herein attached via oxygen linkage to the rest of the molecule, i.e., of the general formula —OR¹, wherein R¹ is an alkyl, cycloalkyl, or aromatic group as defined herein, e.g., —OCH₃, —OC₂H₅, or —OC₆H₅ which may be substituted or unsubstituted, and the like.

Representative examples of alkyl groups for use herein include, by way of example, a linear or branched hydrocarbon chain radical containing carbon and hydrogen atoms of from 1 to about 30 carbon atoms or from 1 to 12 carbon atoms or from 1 to 6 carbon atoms with or without unsaturation, to the rest of the molecule, e.g., methyl, ethyl, n-propyl, 1-methylethyl (isopropyl), n-butyl, n-pentyl, etc., and the like.

Representative examples of alkenyl groups for use herein include, by way of example, a straight or branched hydrocarbon chain radical containing from about 3 to about 30 carbon atoms with at least one carbon-carbon double bond such as, for example, propenyl, butenyl, pentenyl and the like.

Representative examples of alkynyl groups for use herein include, by way of example, a straight or branched hydrocarbon chain radical containing from about 3 to about 30 carbon atoms with at least one carbon-carbon triple bond such as, for example, propynyl, butynyl, pentynyl and the like.

Representative examples of cycloalkyl groups for use herein include, by way of example, a substituted or unsubstituted non-aromatic mono or multicyclic ring system of about 3 to about 30 carbon atoms or from 3 to 12 carbon atoms or from 3 to 6 carbon atoms such as, for example, cyclopropyl, cyclobutyl, cyclopentyl, cyclohexyl, perhydronapththyl, adamantyl and norbornyl groups bridged cyclic group or sprirobicyclic groups, e.g., sprio-(4,4)-non-2-yl and the like, optionally containing one or more heteroatoms, e.g., O and N, and the like.

Representative examples of heterocyclic groups for use herein include, by way of example, a substituted or unsubstituted stable 3 to about 15 membered ring radical, containing carbon atoms and from one to five heteroatoms, e.g., nitrogen, phosphorus, oxygen, sulfur and mixtures thereof. Suitable heterocyclic ring radicals for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. Examples of such heterocyclic groups include, but are not limited to, azetidinyl, acridinyl, benzodioxolyl, benzodioxanyl, benzofurnyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, isochromanyl and the like and mixtures thereof.

Representative examples of aryl groups for use herein include, by way of example, a substituted or unsubstituted monoaromatic or polyaromatic radical containing from about 5 to about 30 carbon atoms or from 5 to 12 carbon atoms or from 5 to 8 carbon atoms such as, for example, phenyl, naphthyl, tetrahydronapthyl, indenyl, biphenyl and the like, optionally containing one or more heteroatoms, e.g., O and N, and the like.

Representative examples of heteroaryl groups for use herein include, by way of example, a substituted or unsubstituted stable 5 to about 30 membered monoaromatic or polyaromatic radical, containing carbon atoms and from one to five heteroatoms, e.g., nitrogen, phosphorus, oxygen, sulfur and mixtures thereof.

Representative examples of fused ring polycyclic-aryl-alkyl groups and similar terms such as, fused ring polycyclic-alkyl-aryl groups, fused ring polycyclo-aryl-alkyl groups, and fused ring polycyclo-alkyl-aryl groups means a fused ring polycyclic group that includes at least one aryl ring and at least one cycloalkyl ring that are fused together to form a fused ring structure. For purposes of non-limiting illustration, examples of fused ring polycyclic-aryl-alkyl groups include, but are not limited to indenyl, 9H-fluorenyl, cyclopentanaphthenyl, and indacenyl.

Representative examples of aralkyl groups as used herein, and in accordance with some embodiments, include, but are not limited to, C₆ to C₂₄ aralkyl, such as a C₆ to C₁₀ aralkyl, and means an aryl group substituted with an alkyl group.

Representative examples of amine groups for use herein include, by way of example, an amine of the general formula —R²NR³R⁴ wherein R², R³ and R⁴ are independently hydrogen or a C₁-C₃₀ hydrocarbon such as, for example, alkyl groups, aromatic groups, or cycloalkyl groups as defined herein, and the like.

The term “carbonyl group” as used herein is a divalent group of the formula —C(═O).

In an illustrative embodiment, R and R* are each hydrogen and R** is a (meth)acrylate-containing reactive end group.

In another illustrative embodiment, as may be combined with one or more of the preceding paragraphs, R** is positioned on the aromatic ring in the para position relative to the OH moiety.

The foregoing UV blockers for use herein are known and either commercially available from such sources as, for example, Aldrich, Polysciences, Gelest, and Melrob, or can be made by methods within the purview of one skilled in the art.

Representative examples of suitable UV blockers include 2-(2′-hydroxy-3′-methallyl-5′-methylphenyl)benzotriazole, commercially available as o-Methallyl Tinuvin P (“oMTP”) from Polysciences, Inc., Warrington, Pa., 3-(2H-benzo[d][1,2,3]triazol-2-yl)-4-hydroxyphenylethyl methacrylate, and 2-(3-(tert-butyl)-4-hydroxy-5-(5-methoxy-2H-benzo[d][1,2,3]triazol-2-yl) phenoxy)ethyl methacrylate.

Suitable UV blockers include, for example, one or more compounds of the following formulae:

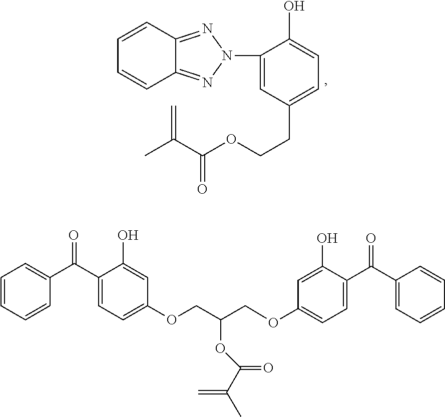 [SMILES: C=C(C)C(=O)OCCc1ccc(O)c(-n2nc3ccccc3n2)c1 ; C=C(C)C(=O)OC(COc1ccc(C(=O)c2ccccc2)c(O)c1)COc1ccc(C(=O)c2ccccc2)c(O)c1]

(2-Propenoic acid, 2-methyl,2-(4-benzoyl-3-hydroxyphenoxy)-1-[(4-benzoyl3-hydroxyphenoxy)methyl ester),

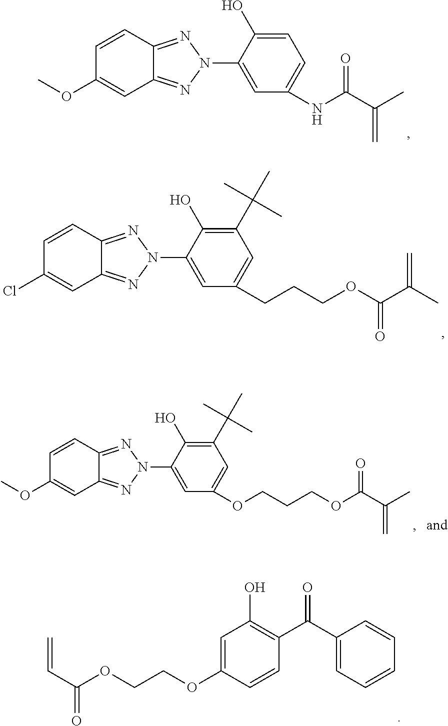

The foregoing UV blockers are merely illustrative and not intended to be limiting. Any known UV blocker comprising a phenolic group having a hydroxyl moiety and one or more ethylenically unsaturated reactive groups or later developed UV blocker comprising a phenolic group having a hydroxyl moiety and one or more ethylenically unsaturated reactive groups are contemplated for use herein.

The polymerization composition can contain from about 0.1 wt. % to about 5 wt. %, based on the total weight of the polymerization composition, of a UV blocker. In some embodiments, the polymerization composition can contain from about 0.5 wt. % to about 1.5 wt. %, based on the total weight of the polymerization composition, of a UV blocker.

In non-limiting illustrative embodiments, as may be combined with one or more of the preceding paragraphs, the polymerization composition can further include one or more blue-light blockers having an ethylenically unsaturated reactive group. Many reactive blue-light absorbing compounds are known, see, e.g., the reactive blue-light absorbing compounds described in U.S. Pat. Nos. 5,470,932; 8,207,244; and 8,329,775, the contents of which are hereby incorporated by reference.

In non-limiting illustrative embodiments, a class of blue light blockers can include a phenolic group comprising a hydroxyl moiety and one or more ethylenically unsaturated reactive groups represented by an acridone compound having a structure of Formula V:

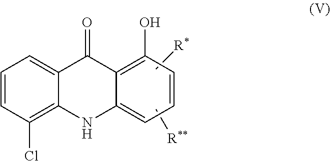

wherein R* and R** are as defined above.

In an illustrative embodiment, R* is hydrogen and R** is a (meth)acrylate-containing reactive end group as defined above.

In some embodiments, R** is positioned on the aromatic ring in the para position relative to the OH moiety.

The foregoing blue light blockers for use herein are known and either commercially available from such sources as, for example, Vishwa-Syntharo PharmaCompany, or can be made by methods within the purview of one skilled in the art.

The foregoing one or more blue light blockers comprising a phenolic group having a hydroxyl moiety and one or more ethylenically unsaturated reactive groups are merely illustrative and not intended to be limiting. Any known blue light blockers comprising a phenolic group having a hydroxyl moiety and one or more ethylenically unsaturated reactive groups or later developed blue light blockers comprising a phenolic group having a hydroxyl moiety and one or more ethylenically unsaturated reactive groups are contemplated for use herein.

In some embodiments, a blue-light blocker is N-2-[3-(2′-methylphenylazo)-4-hydroxyphenyl]ethyl methacrylamide.

The blue-light absorbers can be present in the polymerization composition in an amount ranging from about 0.005 wt. % to about 1 wt. %, based on the total weight of the polymerization composition. In another illustrative embodiment, the blue-light absorbers can be present in the polymerization composition in an amount ranging from about 0.01 wt. % to about 1 wt. %, based on the total weight of the polymerization composition.

In an illustrative embodiment, as may be combined with one or more of the preceding paragraphs, a copolymerization product of a polymerization composition comprising (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group, (v) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, and optionally (vi) an ultraviolet blocker having an ethylenically unsaturated reactive group and/or (vii) a blue-light blocker having an ethylenically unsaturated reactive group can be prepared using free radical polymerization techniques with the structure of the polymer being completely random or controlled by the reactivity ratios of the respective monomers.

In some embodiments, a random copolymer can be obtained by (1) mixing the components to obtain a polymerization composition, (2) adding a polymerization initiator to the polymerization composition, and (3) subjecting the monomer/initiator mixture to a source of heat or radiation such as ultraviolet light, visible light, or high energy radiation to obtain a copolymerization product as discussed below. Suitable free radical thermal polymerization initiators include acetyl peroxide, lauroyl peroxide, decanoyl peroxide, coprylyl peroxide, benzoyl peroxide, tertiary butyl peroxypivalate, sodium percarbonate, tertiary butyl peroctoate, and azobis-isobutyronitrile (AIBN), and the like. Representative UV initiators are those known in the art and include benzoin methyl ether, benzoin ethyl ether, Darocure® 1173, 1164, 2273, 1116, 2959, 3331 (EM Industries) and Irgacure® 651 and 184 (Ciba-Geigy), 2,2′Azobis(2-methylpropionitrile) (Vazo 64) and the like. Generally, the initiator will be employed in the mixture at a concentration of about 0.01 to about 5 percent by weight of the total mixture.

Suitable polymerization conditions include, for example, a temperature of between about 60° C. to about 100° C. for a time period of about 30 minutes to about 48 hours. If desired, the reaction can be carried out in the presence of a suitable solvent. Suitable solvents are in principle all solvents which dissolve the monomers used including, for example, 1,4-dioxane, hexanol, dimethylformamide; acetone, cyclohexanone, toluene, and the like and mixtures thereof.

The copolymerization product disclosed herein can also be prepared using techniques of controlled radical polymerization, e.g., by reversible addition-fragmentation chain transfer (RAFT) polymerization or atom-transfer radical polymerization (ATRP) employing a chain transfer agent that allows construction of copolymers with a well-defined molecular weight distribution and narrow polydispersity. RAFT polymerization is particularly preferred because it is compatible with a wide variety of vinyl monomers.

In non-limiting illustrative embodiments, the RAFT agents suitable for use herein can be based upon thio carbonyl thio chemistry which is well known to those of ordinary skill in the art. The thio carbonyl thio fragment can be derived from a RAFT agent such as, for example, a xanthate-containing compound, trithiocarbonate-containing compound, dithiocarbamate-containing compound, a dithiobenzoate-containing compound or dithio ester-containing compound, wherein each compound contains a thio carbonyl thio group. One class of RAFT agents that can be used herein is of the general formula:

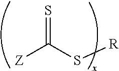

wherein x is 1 or 2, Z is a substituted oxygen (e.g., xanthates (—O—R)), a substituted nitrogen (e.g., dithiocarbamates (—NRR)), a substituted sulfur (e.g., trithiocarbonates (—S—R)), a dithiobenzoate, a substituted or unsubstituted C₁-C₂₀ alkyl or C₃-C₂₅ unsaturated, or partially or fully saturated ring (e.g., dithioesters (—R)) or carboxylic acid-containing group; and R is independently a straight or branched, substituted or unsubstituted C₁-C₃₀ alkyl cyano group, a straight or branched, substituted or unsubstituted C₁-C₃₀ alkyl group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a C₁-C₂₀ ester group; an ether or polyether-containing group; an alkyl- or arylamide group; an alkyl- or arylamine group; a substituted or unsubstituted C₅-C₃₀ heteroaryl group; a substituted or unsubstituted C₃-C₃₀ heterocyclic ring; a substituted or unsubstituted C₄-C₃₀ heterocycloalkyl group; a substituted or unsubstituted C₆-C₃₀ heteroarylalkyl group; and combinations thereof.

The substituents in the ‘substituted oxygen’, ‘substituted nitrogen’, ‘substituted sulfur’, ‘substituted alkyl’, ‘substituted alkylene, ‘substituted cycloalkyl’, ‘substituted cycloalkylalkyl’, ‘substituted cycloalkenyl’, ‘substituted arylalkyl’, ‘substituted aryl’, ‘substituted heterocyclic ring’, ‘substituted heteroaryl ring,’ ‘substituted heteroarylalkyl’, ‘substituted heterocycloalkyl ring’, ‘substituted cyclic ring’ may be the same or different and include one or more substituents such as hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocycloalkyl ring, substituted or unsubstituted heteroarylalkyl, substituted or unsubstituted heterocyclic ring, and the like.

Representative examples of RAFT agents for use herein include, but are not limited to, 4-cyano-4-(dodecyl-sulfanylthiocarbonyl)sulfanylpentanoic acid, S-cyanomethyl-5-dodecyltrithiocarbonate, S-(2-cyano-2-propyl)-S-dodecyltrithiocarbonate, 3-benzylsulfanylthiocarbonylsulfanyl-propionic acid, cumyl dithiobenzoate, 2-cyanoprop-2-yl dithiobenzoate (i.e., cyanoisopropyl dithiobenzoate), 4-thiobenzoylsulfanyl-4-cyanopentanoic acid (TCA), S,S′-bis(α,α′-dimethyl-alpha″-acetic acid)-trithiocarbonate (BATC), benzyl dodecyl trithiocarbonate, ethyl-2-dodecyl trithiocarbony) proprionate, S-sec propionic acid O-ethyl xanthate, α-ethyl xanthylphenylacetic acid, ethyl α-(o-ethyl xanthyl) proprionate, ethyl α-(ethyl xanthyl) phenyl acetate, ethyl 2-(dodecyl trithiocarbonyl) phenyl acetate, ethyl 2-(dodecyl trithiocarbonyl) propionate, 2-(dodecylthiocarbonylthiol) propanoic acid, and the like and mixtures thereof.

There is no particular limitation on the organic chemistry used to form the RAFT agent and is within the purview of one skilled in the art.

The copolymerization product disclosed herein can be obtained in by (1) mixing an alkylacrylamide monomer, a hydroxyalkyl acrylate monomer, a non-bulky organosilicon-containing monomer having an ethylenically unsaturated reactive group, a bulky siloxane monomer having an ethylenically unsaturated reactive group, and one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group with a RAFT agent and a polymerization initiator, and (2) subjecting the monomer/RAFT agent/initiator mixture to a source of heat. Suitable initiators include, for example, free-radical-generating polymerization initiators of the type illustrated by acetyl peroxide, lauroyl peroxide, decanoyl peroxide, coprylyl peroxide, benzoyl peroxide, tertiary butyl peroxypivalate, sodium percarbonate, tertiary butyl peroctoate, and azobisisobutyronitrile (AIBN).

The reaction can be carried out at a temperature of between about 50° C. to about 80° C. for a time period of about 30 minutes to about 48 hours. If desired, the reaction can be carried out in the presence of a suitable solvent. Suitable solvents are in principle all solvents which dissolve the monomer used, for example, 1,4-dioxane, hexanol, dimethylformamide; acetone, cyclohexanone, toluene, and the like and mixtures thereof.

In an illustrative embodiment, the RAFT procedure is carried using a mixture including (i) from about 2 wt. % to about 40 wt. %, based on the total weight of the mixture, of an alkylacrylamide monomer, (ii) from about 10 wt. % to about 20 wt. %, based on the total weight of the mixture, of a hydroxyalkyl acrylate monomer, (iii) from about 10 wt. % to about 60 wt. %, based on the total weight of the mixture, of a non-bulky organosilicon-containing monomer having an ethylenically unsaturated reactive group, (iv) from about 20 wt. % to about 60 wt. %, based on the total weight of the mixture, of a bulky siloxane monomer having an ethylenically unsaturated reactive group, (v) from about 5 wt. % to about 50 wt. %, based on the total weight of the mixture, of one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group and (vi) from about 0.05 wt. % to about 2 wt. %, based on the total weight of the mixture, of the RAFT agent. The level of initiator employed will vary within the range of 0.01 wt. % to 2 wt. % of the mixture of monomers. In some embodiments, the mixture for the RAFT procedure further includes from about 0.1 wt. % to about 5 wt. %, based on the total weight of the mixture, of an ultraviolet blocker having an ethylenically unsaturated reactive group.

A non-limiting schematic representation of a synthetic method for making a copolymerization product with a RAFT agent is set forth below in Scheme I.

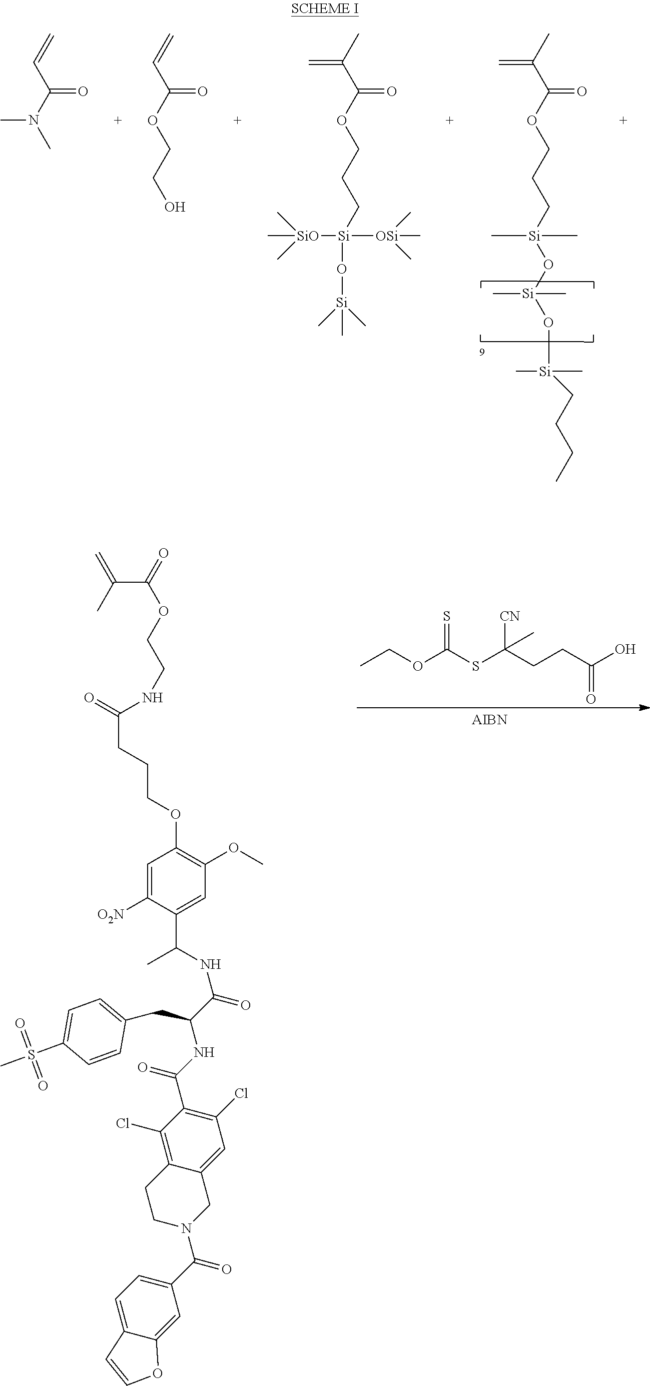

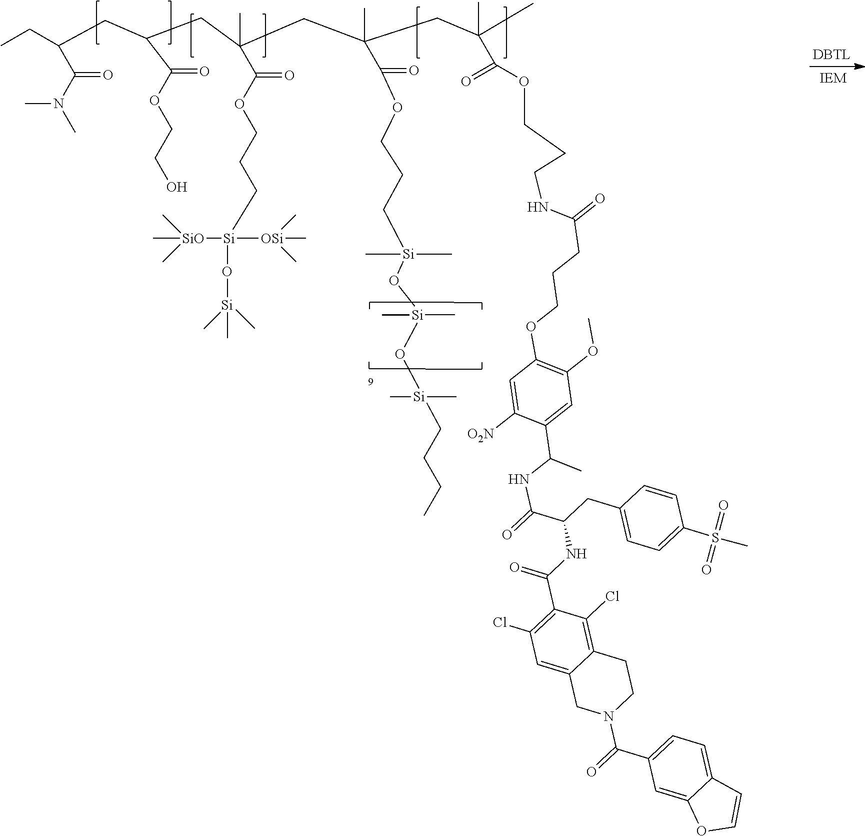

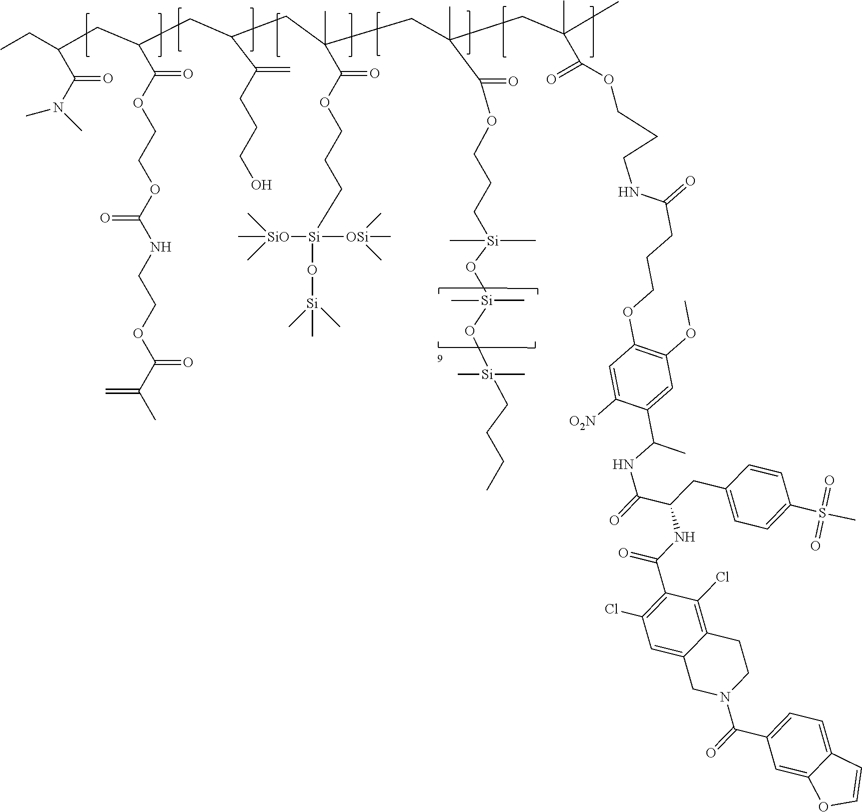

In the case where the copolymerization product disclosed herein is obtained from ATRP polymerization, the ethylenically unsaturated groups may be introduced by appropriate selection of a suitable ATRP initiator or by displacement reactions of the terminal halogen atom. Suitable ATRP groups for use herein include any standard monofunctional or difunctional ATRP group as is well known to those of ordinary skill in the art. A comprehensive review on the use of ATRP initiators or displacement of the terminal halogen using electrophilic, nucleophilic, and radical reactions to produce telechelic polymers is disclosed in, for example, Matyjaszewski, K.; Xia, J. Chem. Rev., 101, 2921-2990 (2001).

In one embodiment, a useful ATRP group includes an ethylenically unsaturated ATRP initiator such as, for example, vinyl functionalized ATRP initiators, e.g., prop-2-enyl-2′-bromoisobutyrate, vinyl chloroacetate, allyl chloroacetate, allyl bromide and the like.

In another embodiment, a useful ATRP group includes a non-ethylenically unsaturated ATRP initiator that can be converted to an ethylenically unsaturated initiator by a subsequent step. Examples of such initiators include α-bromo-isobutyric acid, hydroxyethyl 2-bromopropionate, glycidol 2-bromopropionate, tert-butyl 2-bromopropionate, and 4-bromobenzyl bromide, and the like.

In an illustrative embodiment, the copolymerization product is obtained from ATRP polymerization by (1) mixing an alkylacrylamide monomer, a hydroxyalkyl acrylate monomer, a non-bulky organosilicon-containing monomer having an ethylenically unsaturated reactive group, a bulky siloxane monomer having an ethylenically unsaturated reactive group, one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group and optionally an ultraviolet blocker having an ethylenically unsaturated reactive group and/or a blue-light blocker having an ethylenically unsaturated reactive group with, for example, an ATRP initiator and suitable ATRP catalyst such as a copper (I) bromide and (2) subjecting the monomer/ATRP agent/initiator mixture to a source of heat.

The reaction can be carried out at a temperature of between about 60° C. to about 100° C. for a time period of about 30 minutes to about 48 hours. If desired, the reaction can be carried out in the presence of a suitable solvent. Suitable solvents are in principle all solvents which dissolve the monomer used, for example, 1,4-dioxane, hexanol, dimethylformamide; acetone, cyclohexanone, toluene, and the like and mixtures thereof.

The ATRP procedure is carried using a mixture including (i) from about 0.1 wt. % to about 20 wt. %, based on the total weight of the mixture, of an alkylacrylamide monomer, (ii) from about 5 wt. % to about 20 wt. %, based on the total weight of the mixture, of a hydroxyalkyl acrylate monomer, (iii) from about 10 wt. % to about 20 wt. %, based on the total weight of the mixture, of a non-bulky organosilicon-containing monomer having an ethylenically unsaturated reactive group, (iv) from about 20 wt. % to about 60 wt. %, based on the total weight of the mixture, of a bulky siloxane monomer having an ethylenically unsaturated reactive group, (v) from about 5 wt. % to about 50 wt. %, based on the total weight of the mixture, of one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, and (vi) from about 0.05 wt. % to about 5 wt. %, based on the total weight of the mixture, of the ATRP initiator. The level of catalyst employed will vary within the range of 0.01 wt. % to 2 wt. % of the mixture of monomers. In some embodiments, the mixture for the ATRP procedure further includes from about 20 to about 40 wt. %, based on the total weight of the mixture, of an ultraviolet blocker having an ethylenically unsaturated reactive group.

As one skilled in the art will readily appreciate, the copolymerization product disclosed herein can contain a balance of monomeric units derived from the alkylacrylamide monomer, monomeric units derived from the hydroxyalkyl acrylate monomer, monomeric units derived from the non-bulky organosilicon-containing monomer having an ethylenically unsaturated reactive group, monomeric units derived from the bulky siloxane monomer having an ethylenically unsaturated reactive group, monomeric units derived from one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group and monomeric units derived from an ultraviolet blocker having an ethylenically unsaturated reactive group.

In some embodiments, a copolymerization product can include from about 50 to about 400 repeating units of monomeric units derived from the alkylacrylamide monomer, from about 50 to about 200 repeating units of monomeric units derived from the hydroxyalkyl acrylate monomer, from about 20 to about 300 repeating units of monomeric units derived from the non-bulky organosilicon-containing monomer having an ethylenically unsaturated reactive group, from about 1 to about 400 repeating units of monomeric units derived from the bulky siloxane monomer having an ethylenically unsaturated reactive group, (v) from about 5 to about 50 repeating units of monomeric units derived from the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group, and optionally from about 1 to about 25 repeating units of monomeric units derived from an ultraviolet blocker having an ethylenically unsaturated reactive group.

In one or more additional non-limiting illustrative embodiments, as may be combined with one or more of the preceding paragraphs, the copolymerization product is thereafter reacted with a monomer having a reactive functionality complementary to the hydroxyl moiety of the hydroxyalkyl acrylate monomer and a polymerizable ethylenically unsaturated reactive group to obtain a silicone prepolymer containing lifitegrast or salt thereof-containing monomer or polymer. The foregoing reaction will result in additional monomeric units derived from the hydroxyalkyl acrylate monomer with the hydroxy moiety being reacted with the reactive functionality of the monomer such that the hydroxyalkyl acrylate monomer contains an end functionalized group, i.e., a polymerizable ethylenically unsaturated reactive group which can be complementary to an ethylenically unsaturated reactive group of a contact lens-forming comonomer as discussed below. Suitable polymerizable ethylenically unsaturated reactive groups can be any of those discussed above.

In an illustrative embodiment, a polymerizable ethylenically unsaturated reactive group can be one or more of an acrylate end group and a methacrylate end group.

A suitable monomer having a reactive functionality complementary to the hydroxyl moiety of the hydroxyalkyl acrylate monomer and a polymerizable ethylenically unsaturated reactive group include, for example, 2-isocyanatoethyl acrylate, 3-isocyanatopropyl acrylate, 2-isocyanatoethyl methacrylate, 1-methyl-2-isocyanatoethyl methacrylate, 1,1-dimethyl-2-isocyanatoethyl acrylate, (meth)acryloyl chloride, vinyl chloroformate and the like.

The copolymerization product is present in the reaction mixture in an amount ranging from about 10 wt. % to about 90 wt. %, based on the total weight of the reaction mixture. In an illustrative embodiment, the copolymerization product is present in the reaction mixture in an amount ranging from about 50 wt. % to about 80 wt. %, based on the total weight of the reaction mixture.

The monomer having a reactive functionality complementary to the hydroxyl moiety of the hydroxyalkyl acrylate and a polymerizable ethylenically unsaturated reactive group is present in the reaction mixture in an amount ranging from about 5 wt. % to about 60 wt. %, based on the total weight of the reaction mixture. In an illustrative embodiment, the monomer having a reactive functionality complementary to one of the one or more reactive functionalities of the hydrophilic monomer and a polymerizable ethylenically unsaturated reactive group is present in the reaction mixture in an amount ranging from about 10 wt. % to about 20 wt. %, based on the total weight of the reaction mixture.

The reaction of the copolymerization product and the monomer having a reactive functionality complementary to the hydroxyl moiety of the hydroxyalkyl acrylate and a polymerizable ethylenically unsaturated reactive group can be carried out in the presence of a catalyst. Suitable catalysts include, for example, the stannous salts of carboxylic acids, such as stannous octoate, stannous oleate, stannous acetate, and stannous laurate, dialkyltin dicarboxylates, such as dibutyltin dilaurate and dibutyltin diacetate which are known in the art as urethane catalysts, as are tertiary amines and tin mercaptides. The amount of catalyst employed is generally between about 0.01 wt. % to about 5 wt. % of the mixture catalyzed.

The reaction can be carried out at a temperature of between about 60° C. to about 100° C. for about 2 hours to about 24 hours. The reaction can be carried out in the presence of a suitable solvent as discussed above.

Accordingly, in a non-limiting illustrative embodiment, a silicone prepolymer disclosed herein comprises:
(i) monomeric units derived from the alkylacrylamide monomer,
(ii) monomeric units derived from the hydroxyalkyl acrylate monomer,
(iii) monomeric units derived from the hydroxyalkyl acrylate monomer where the acrylate moiety is attached to the backbone of the copolymer and the hydroxy moiety is end functionalized with the monomer having a reactive functionality complementary to the hydroxyl moiety of the hydroxyalkyl acrylate and a polymerizable ethylenically unsaturated reactive group,
(iv) monomeric units derived from the bulky siloxane monomer,
(v) monomeric units derived from the non-bulky siloxane monomer,
(v) monomeric units derived from one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, and
optionally (vi) monomeric units derived from an ultraviolet blocker having one or more reactive functionalities.

In some embodiments, a silicone prepolymer is a silicone random copolymer.

In some embodiments, a silicone prepolymer disclosed herein includes:
from about 50 to about 400 repeating units of monomeric units derived from the alkylacrylamide monomer,
from about 50 to about 200 repeating units of monomeric units derived from the hydroxyalkyl acrylate monomer,
from about 5 to about 20 repeating units of monomeric units derived from the hydroxyalkyl acrylate monomer where the acrylate moiety is attached to the backbone of the copolymer and the hydroxy moiety is end functionalized with the monomer having a reactive functionality complementary to the hydroxyl moiety of the hydroxyalkyl acrylate and a polymerizable ethylenically unsaturated reactive group,
from about 20 to about 300 repeating units of monomeric units derived from the non-bulky organosilicon-containing monomer having an ethylenically unsaturated reactive group,
from about 1 to about 400 repeating units of monomeric units derived from the bulky siloxane monomer having an ethylenically unsaturated reactive group, and
from about 1 to about 50 repeating units of monomeric units derived from the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group.

In non-limiting illustrative embodiments, as may be combined with one or more of the preceding paragraphs, a silicone prepolymer disclosed herein further includes from about 1 to about 25 repeating units of the monomeric units derived from an ultraviolet blocker having an ethylenically unsaturated reactive group.

In some embodiments, a silicone prepolymer disclosed herein can have a weight average molecular weight ranging from about 2,000 to about 300,000 Da.

In non-limiting illustrative embodiments, as may be combined with one or more of the preceding paragraphs, a silicone contact lens disclosed herein is formed from a polymerization product of a silicone contact lens-forming mixture comprising one or more of the silicone prepolymers disclosed herein as discussed below.

In a non-limiting illustrative embodiment, a silicone contact lens disclosed herein can have an oxygen permeability of at least about 50 Barrers. In some embodiments, a silicone contact lens disclosed herein can have an oxygen permeability of from about 75 Barrers to about 175 Barrers.

In a non-limiting illustrative embodiment, a silicone contact lens disclosed herein can have an equilibrium water content of at least about 20 wt. %. In another illustrative embodiment, a silicone contact lens can have an equilibrium water content of from about 20 wt. % to about 60 wt. %.

In some embodiments, a silicone contact lens disclosed herein demonstrates sufficient blocking of UV light to meet both FDA Class I and II specifications for UV blocking. As stated above, Class I contact lenses must block more than 90% of UVA i.e., 316 to 380 nm, radiation and 99% of UVB, i.e., 280 to 315 nm, radiation. Class II contact lenses must block more than 50% of UVA and 95% of UVB radiation.

In some embodiments, a silicone prepolymer disclosed herein is present in the silicone contact lens-forming mixture in an amount ranging from about 50 wt. % to about 100 wt. %, based on the total weight of the silicone contact lens-forming mixture. In some embodiments, a silicone prepolymer disclosed herein is present in the silicone contact lens-forming mixture in an amount ranging from about 60 wt. % to about 80 wt. %, based on the total weight of the silicone contact lens-forming mixture.

In non-limiting illustrative embodiments, as may be combined with one or more of the preceding paragraphs, the silicone contact lens-forming mixture for forming the silicone contact lens disclosed herein can further contain one or more contact lens-forming monomers. For example, the silicone contact lens-forming mixture further comprises one or more silicone contact lens-forming monomers, one or more hydrophilic comonomers, or both.

In some embodiments, the one or more contact lens-forming monomers include one or more silicone contact lens-forming monomers. For example, one class of silicone contact lens-forming monomers includes one or more monofunctional silicone monomers represented by a structure of Formula V:

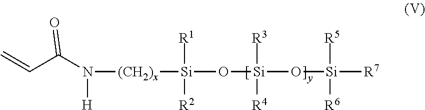

wherein R¹, R², R³ and R⁴ are independently hydrogen, an alkyl group, a halo alkyl group, a cycloalkyl group, a heterocycloalkyl group, an alkenyl group, a haloalkenyl group, an aryl group and a heteroaryl group; R⁵, R⁶ and R⁷ are independently a straight or branched alkyl group; x is from 1 to 6; and y is from 3 to 15.

In one embodiment, R¹, R², R³ and R⁴ are independently hydrogen, a C₁ to C₁₂ alkyl group, a C₁ to C₁₂ halo alkyl group, a C₃ to C₁₂ cycloalkyl group, a C₃ to C₁₂ heterocycloalkyl group, a C₂ to C₁₂ alkenyl group, a C₂ to C₁₂ haloalkenyl group, a C₆ to C₁₂ aromatic group and a C₆ to C₁₂ heteroaromatic group; R⁵, R⁶ and R⁷ are independently a straight or branched C₁ to C₁₂ alkyl group; x is from 1 to 6; and y is from 3 to 8.

In one embodiment, R¹, R², R³ and R⁴ are independently hydrogen or a C₁ to C₆ alkyl group; R⁵, R⁶ and R⁷ are independently a straight or branched C₁ to C₆ alkyl group; x is from 1 to 6; and y is from 3 to 8.

In one embodiment, R¹, R², R³ and R⁴ are independently a C₁ to C₃ alkyl group; R⁵ and R⁶ are independently a C₁ to C₃ alkyl group; R⁷ is a straight or branched C₃ to C₆ alkyl group; x is from 2 to 4; and y is from 3 to 8.

In some embodiments, R¹, R², R³ and R⁴ are independently a C₁ to C₃ alkyl group; R⁵ and R⁶ are independently a C₁ to C₃ alkyl group; R⁷ is a straight or branched C₃ to C₆ alkyl group; x is from 2 to 4; and y is from 3 to 15.

Representative examples of alkyl groups, cycloalkyl groups and aryl groups can any of those discussed above.

Representative examples of cycloalkylalkyl groups for use herein include, by way of example, a substituted or unsubstituted, cyclic ring-containing radical containing from about 4 to about 30 carbon atoms or from 3 to about 6 carbon atoms directly attached to the alkyl group which are then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like to form a heterocycloalkylalkyl group.

Representative examples of cycloalkenyl groups for use herein include, by way of example, a substituted or unsubstituted cyclic ring-containing radical containing from about 3 to about 30 carbon atoms or from 3 to about 6 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like to form a heterocycloalkenyl group.

In an illustrative embodiment, as may be combined with one or more of the preceding paragraphs, the one or more silicone contact lens-forming monomers can include, as a class of representative silicone contact lens-forming monomers, one or more non-bulky organosilicon-containing monomers. The one or more non-bulky organosilicon-containing monomers can be any of those discussed above.

In an illustrative embodiment, as may be combined with one or more of the preceding paragraphs, the one or more silicone contact lens-forming monomers can include, as a class of representative silicone contact lens-forming monomers, one or more bulky siloxane monomers. The one or more bulky siloxane monomers can be any of those discussed above.

In accordance with one or more additional non-limiting illustrative embodiments, as may be combined with one or more of the preceding paragraphs, the one or more silicone contact lens-forming monomers can be present in the silicone contact lens-forming mixture in an amount ranging from about 10 wt. % to about 50 wt. %, based on the total weight of the silicone contact lens-forming mixture.

In some embodiments, the one or more contact lens-forming monomers include one or more hydrophilic comonomers. Suitable one or more hydrophilic comonomers include, for example, unsaturated carboxylic acids, acrylamides, vinyl lactams, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof.

Representative examples of unsaturated carboxylic acids include, but are not limited to, methacrylic acid, acrylic acid and the like and mixtures thereof. Representative examples of acrylamides include, but are not limited to, alkylamides such as N,N-dimethylacrylamide, N,N-dimethylmethacrylamide and the like and mixtures thereof. Representative examples of cyclic lactams include, but are not limited to, N-vinyl-2-pyrrolidone, N-vinyl caprolactam, N-vinyl-2-piperidone and the like and mixtures thereof. Representative examples of hydroxyl-containing (meth)acrylates include, but are not limited to, 2-hydroxyethyl methacrylate (HEMA), glycerol methacrylate and the like and mixtures thereof. Additional device-forming hydrophilic comonomers include, for example, the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable device-forming hydrophilic comonomers will be apparent to one skilled in the art. Mixtures of the foregoing device-forming hydrophilic comonomers can also be used in the silicone contact lens-forming mixtures herein.

The silicone contact lens-forming mixture may further include one or more additional contact lens-forming monomers including, for example, a copolymerizable group and a reactive functional group. The copolymerizable group is preferably an ethylenically unsaturated group, such that this contact lens-forming monomer copolymerizes with the hydrophilic monomer and any other contact lens-forming monomers in the initial silicone contact lens-forming mixture. Additionally, the one or more additional contact lens-forming monomers can include a reactive functional group that reacts with a complementary reactive group of the silicone prepolymer.

In an embodiment, reactive groups of the one or more additional contact lens-forming monomers include epoxide groups. Accordingly, the one or more additional contact lens-forming monomers are those that include both an ethylenically unsaturated group (that permits the monomer to copolymerize with the hydrophilic monomer) and the epoxide group (that does not react with the hydrophilic comonomers but remains to react with the silicone prepolymer. Suitable one or more additional contact lens-forming monomers include, for example, glycidyl methacrylate, glycidyl acrylate, glycidyl vinylcarbonate, glycidyl vinylcarbamate, and 4-vinyl-1-cyclohexene-1,2-epoxide.

The one or more contact lens-forming monomers such as one or more hydrophilic comonomers can be present in the silicone contact lens-forming mixture in an amount ranging from about 10 wt. % to about 50 wt. %, based on the total weight of the silicone contact lens-forming mixture. In some embodiments, the one or more contact lens-forming monomers can be present in the silicone contact lens-forming mixture in an amount ranging from about 20 wt. % to about 60 wt. %, based on the total weight of the silicone contact lens-forming mixture.

The silicone contact lens-forming mixtures disclosed herein may further contain, as necessary and within limits not to impair the purpose and effect of the illustrative embodiments disclosed herein, various additives such as an antioxidant, coloring agent, toughening agents and the like and other constituents as is well known in the art.

The silicone contact lenses of the illustrative embodiments can be prepared by polymerizing the foregoing silicone contact lens-forming mixture to form a product that can be subsequently formed into the appropriate shape by, for example, lathing, injection molding, compression molding, cutting and the like. For example, in producing contact lenses, the initial mixture may be polymerized in tubes to provide rod-shaped articles, which are then cut into buttons. The buttons may then be lathed into contact lenses.

Alternately, the silicone contact lenses may be cast directly in molds, e.g., polypropylene molds, from the silicone contact lens-forming mixtures, e.g., by spincasting and static casting methods. Spincasting methods are disclosed in U.S. Pat. Nos. 3,408,429 and 3,660,545, and static casting methods are disclosed in U.S. Pat. Nos. 4,113,224, 4,197,266, and 5,271,875. Spincasting methods involve charging the silicone contact lens-forming mixtures mixtures to be polymerized to a mold, and spinning the mold in a controlled manner while exposing the mixture to a radiation source such as UV light. Static casting methods involve charging the silicone contact lens-forming mixture between two mold sections, one mold section shaped to form the anterior lens surface and the other mold section shaped to form the posterior lens surface, and curing the silicone contact lens-forming mixture while retained in the mold assembly to form a contact lens, for example, by free radical polymerization of the silicone contact lens-forming mixture.

Examples of free radical reaction techniques to cure the silicone contact lens-forming mixture include thermal radiation, infrared radiation, electron beam radiation, gamma radiation, ultraviolet (UV) radiation, and the like; or combinations of such techniques may be used. U.S. Pat. No. 5,271,875 describes a static cast molding method that permits molding of a finished lens in a mold cavity defined by a posterior mold and an anterior mold. As an additional method, U.S. Pat. No. 4,555,732 discloses a process where an excess of a mixture is cured by spincasting in a mold to form a shaped article having an anterior lens surface and a relatively large thickness, and the posterior surface of the cured spincast article is subsequently lathed to provide a contact lens having the desired thickness and posterior lens surface.

Polymerization may be facilitated by exposing the silicone contact lens-forming mixture to heat and/or radiation, such as ultraviolet light, visible light, or high energy radiation. A polymerization initiator may be included in the mixture to facilitate the polymerization step. Representative examples of free radical thermal polymerization initiators include organic peroxides such as acetyl peroxide, lauroyl peroxide, decanoyl peroxide, stearoyl peroxide, benzoyl peroxide, tertiarylbutyl peroxypivalate, peroxydicarbonate, and the like. Representative UV initiators are those known in the art and include benzoin methyl ether, benzoin ethyl ether, Darocure® 1173, 1164, 2273, 1116, 2959, 3331 (EM Industries) and Irgacure® 651, 184 and 2959 (Ciba-Geigy), 2,2′ Azobis(2-methylpropionitrile) (VAZO 64) and the like. Generally, the initiator will be employed in the silicone contact lens-forming mixture at a concentration of about 0.01 wt. % to about 5 wt. %, based on the total wight of the silicone contact lens-forming mixture.

Polymerization is generally performed in a reaction medium, such as, for example, a solution or dispersion using a solvent, e.g., water or an alkanol containing from 1 to 4 carbon atoms such as methanol, ethanol or propan-2-ol. Alternatively, a mixture of any of the above solvents may be used.

Generally, polymerization can be carried out for about 15 minutes to about 72 hours, and under an inert atmosphere of, for example, nitrogen or argon. If desired, the resulting polymerization product can be dried under vacuum, e.g., for about 5 hours to about 72 hours or left in an aqueous solution prior to use.

Polymerization of the silicone contact lens-forming mixtures will yield a polymerization product, that when hydrated, preferably forms a hydrogel. When producing a hydrogel lens, the silicone contact lens-forming mixture may further include at least a diluent that is ultimately replaced with water when the polymerization product is hydrated to form a hydrogel. The amount of diluent used should be less than about 50 wt. %, and in most cases, the diluent content will be less than about 30 wt. %. However, in a particular polymer system, the actual limit will be dictated by the solubility of the various monomers in the diluent. In order to produce an optically clear copolymer, it is important that a phase separation leading to visual opacity does not occur between the comonomers and the diluent, or the diluent and the final copolymer.

Furthermore, the maximum amount of diluent which may be used will depend on the amount of swelling the diluent causes the final polymers. Excessive swelling will or may cause the copolymer to collapse when the diluent is replaced with water upon hydration. Suitable diluents include, but are not limited to, ethylene glycol, glycerine, liquid poly(ethylene glycol), alcohols, alcohol/water mixtures, ethylene oxide/propylene oxide block copolymers, low molecular weight linear poly(2-hydroxyethyl methacrylate), glycol esters of lactic acid, formamides, ketones, dialkylsulfoxides, butyl carbitol, and the like and mixtures thereof.

If necessary, it may be desirable to remove residual diluent from the contact lens before edge-finishing operations which can be accomplished by evaporation at or near ambient pressure or under vacuum. An elevated temperature can be employed to shorten the time necessary to evaporate the diluent. The time, temperature and pressure conditions for the solvent removal step will vary depending on such factors as the volatility of the diluent and the specific monomeric components, as can be readily determined by one skilled in the art. If desired, the mixture used to produce the hydrogel lens may further include crosslinking and wetting agents known in the prior art for making hydrogel materials.

The silicone contact lenses obtained herein may be subjected to optional machining operations. For example, the optional machining steps may include buffing or polishing a lens edge and/or surface. Generally, such machining processes may be performed before or after the product is released from a mold part, e.g., the lens is dry released from the mold by employing vacuum tweezers to lift the lens from the mold, after which the lens is transferred by means of mechanical tweezers to a second set of vacuum tweezers and placed against a rotating surface to smooth the surface or edges. The silicone contact lens may then be turned over in order to machine the other side of the lens.

The silicone contact lens may then be transferred to individual lens packages containing a buffered saline solution. The saline solution may be added to the package either before or after transfer of the lens. Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, autoclaving.

As one skilled in the art will readily appreciate other steps may be included in the molding and packaging process described above. Such other steps can include, for example, coating the formed lens, surface treating the lens during formation (e.g., via mold transfer), inspecting the lens, discarding defective lenses, cleaning the mold halves, reusing the mold halves, and the like and combinations thereof.

The user can then place the silicone contact lens on the eye during use. As mentioned above, depending on the particular releasable linking group, the lifitegrast or salt thereof will be released on the eye in a sustained release manner thereby providing therapeutic relief for dry eye conditions. For example, the particular releasable linking group can be attached to the lifitegrast or salt thereof-containing monomer or lifitegrast or salt thereof-containing polymer via a pH-labile bond, an acid-labile bond, a base-labile bond, an oxidatively labile bond, a metabolically labile bond, a biochemically labile bond, an enzyme-labile bond, a chemical-labile bond, or a photo-labile bond. Accordingly, the lifitegrast or salt thereof may be released from the silicone contact lens upon exposure to, e.g., light such as UV, near UV or visible light, or the natural tears in the eye or the pH conditions in the eye.

The following examples are provided to enable one skilled in the art to practice the invention and are merely illustrative of the invention. The examples should not be read as limiting the scope of the invention as defined in the claims.

In the examples, the following abbreviations are used.
DMA: N,N-dimethylacrylamide.
NVP: N-vinyl-2-pyrrolidone.
HEMA: 2-hydroxyethyl methacrylate.
EGDMA: Ethylene glycol dimethacrylate.
IMVT: 1,4-bis(4-(2-methacryloxyethyl)phenylamino) anthraquinone.
TrisMA: tris(trimethylsiloxy)silylpropyl methacrylate.
UV416:2-(4-Benzoyl-3-hydroxyphenoxy)ethyl acrylate.
IEM: 2-isocyanatoethyl methacrylate.
DBTDL: Dibutyltin dilaurate.
HEMA-lifitegrast: A monomer having the following structure:

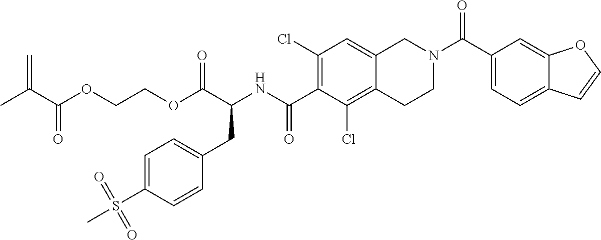 [SMILES: C=C(C)C(=O)OCCOC(=O)[C@H](Cc1ccc(S(C)(=O)=O)cc1)NC(=O)c1c(Cl)cc2c(c1Cl)CCN(C(=O)c1ccc3ccoc3c1)C2]

Ma2D37: A compound having the structure:

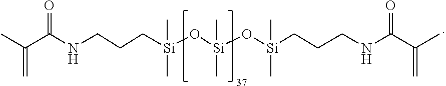

M1EDS6: A compound having the following structure and available from Gelest:

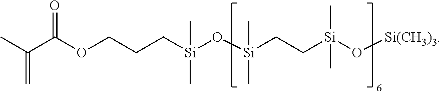

### Example 1

The preparation and functionalization of a silicone prepolymer with lifitegrast is shown below by the general reaction scheme.

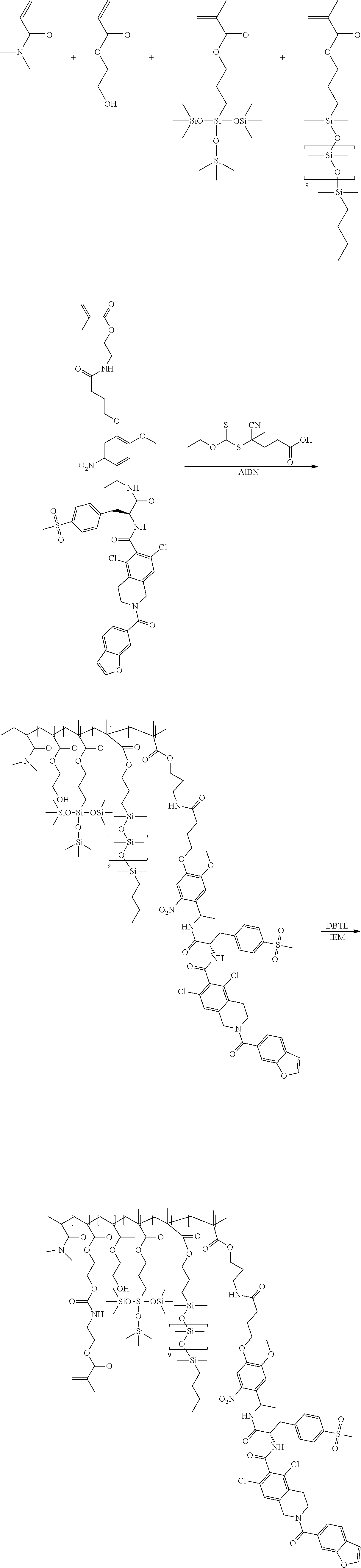

### Experiment Detail:

The monomers, RAFT and AIBN are placed in an air free flask and dissolved in toluene (weight ratio of components:solvent=at least 1:2). The flask is purged with nitrogen for 1 hour, closed and then is placed in a preheated oil bath at 60° C. overnight. The polymer is washed and dried and is then dissolved in toluene, where IEM and DBTDL is added and stirred overnight at room temperature. The polymer is extracted with acetonitrile and dried. Analytical results below.

### Example 2

### Preparation of a Silicone Contact Lens.

A silicone contact lens is prepared using the reaction components listed in Table 1 below, as amounts per weight percent.

TABLE 1

| Component | Wt. % |
|---|---|
| HEMA | 14.89 |
| DMA | 14.89 |
| Silicone Prepolymer of Example 1 | 69.50 |
| Irgacure 819 | 0.69 |
| Tint | 0.02 |

The mixture is made in ambient conditions with no nitrogen or oxygen mitigation. The copolymer is dissolved in HEMA and DMA. The mixture is speed mixed for 10 minutes at 2500 RPM, repeated twice until fully dissolved. Next, Irgacure 819 and tint are added to the mixtures and speed mixed for 10 minutes at 2500 RPM and filtered through 0.2 micron syringe. The mixture is cast in a propylene mold, and is cured in 420 nm LED 4 mW light for 10 minutes to produce a lens.

### Example 3

### Preparation of a Silicone Contact Lens.

A silicone contact lens is prepared using the reaction components listed in Table 2 below, as amounts per weight percent.

TABLE 2

| Component | Wt. % |
|---|---|
| Ma2D37 | 6.00 |
| M1EDS6 | 15.00 |
| TRIS-MA | 27.00 |
| DMA | 7.00 |
| HEMA | 1.00 |
| NVP | 34.00 |
| UV-416 | 1.00 |
| EGDMA | 0.10 |
| IMVT | 0.02 |
| Initiator | 0.50 |
| Hexanol | 7.38 |
| HEMA-Lifitegrast | 1.00 |

The mixture is made in ambient conditions with no nitrogen or oxygen mitigation. The copolymer is dissolved in HEMA and DMA. The mixture is speed mixed for 10 minutes at 2500 RPM, repeated twice until fully dissolved. Next, Irgacure 819 and tint are added to the mixtures and speed mixed for 10 minutes at 2500 RPM and filtered through 0.2 micron syringe. The mixture is cast in ultra polypropylene mold, and is cured in 420 nm LED 4 mW light for 10 minutes to produce a lens.

According to an aspect of present disclosure, a silicone prepolymer comprises a reaction product of (a) a copolymerization product of a polymerization composition comprising (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group and (v) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (b) a monomer having a reactive functionality complementary to the hydroxyl moiety of the hydroxyalkyl acrylate monomer and a polymerizable ethylenically unsaturated reactive group.

In non-limiting illustrative embodiments, as may be combined with one or more of the preceding paragraphs, the alkylacrylamide monomer is N,N-dimethylacrylamide, and the hydroxyalkyl acrylate monomer is a hydroxyethyl acrylate monomer.

In non-limiting illustrative embodiments, as may be combined with one or more of the preceding paragraphs, the non-bulky organosilicon-containing monomer is represented by the following structure:

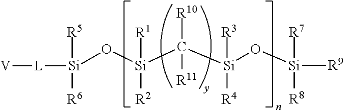

wherein V is an ethylenically unsaturated polymerizable group, L is a linking group or a bond; R¹, R², R³, R⁴, R⁵, R⁶, R⁷, R⁸, and R⁹ are independently hydrogen an alkyl group, a haloalkyl group, a cycloalkyl group, a heterocycloalkyl group, an alkenyl group, a halo alkenyl group, or an aromatic group; R¹⁰ and R¹¹ are independently hydrogen or alkyl, wherein at least one of R¹⁰ and R¹¹ is hydrogen; y is 2 to 7 and n is 1 to 100, or the non-bulky organosilicon-containing monomer is represented by the following structure:

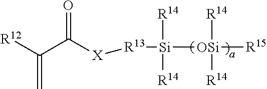

wherein R¹² is H or methyl; X is O or NR¹⁶; wherein R¹⁶ is hydrogen or C₁ to C₄ alkyl, which may be further substituted with one or more hydroxyl groups; R¹³ is a divalent alkyl group, which may further be functionalized with a group selected from the group consisting of an ether group, a hydroxyl group, a carbamate group and combinations thereof; each R¹⁴ is independently a phenyl or C₁ to C₄ alkyl which may be substituted with fluorine, hydroxyl or an ether; R¹⁵ is a C₁ to C₄ alkyl; and a is 2 to 50.

In non-limiting illustrative embodiments, as may be combined with one or more of the preceding paragraphs, the bulky siloxane monomer is selected from the group consisting of a bulky polysiloxanylalkyl (meth)acrylic monomer, a bulky polysiloxanylalkyl carbamate monomer and mixtures thereof.

In non-limiting illustrative embodiments, as may be combined with one or more of the preceding paragraphs, the bulky siloxane monomer is represented by the following structure:

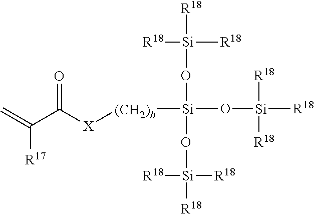

wherein X denotes —O— or —NR¹⁹— where each R¹⁹ is hydrogen or a C₁-C₄ alkyl; R¹⁷ independently denotes hydrogen or methyl; each R¹⁸ independently denotes a C₁-C₆ alkyl radical, a phenyl radical or a group represented by the following structure:

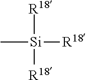

wherein each R^{18′} independently denotes a C₁-C₆ alkyl radical or a phenyl radical; and h is 1 to 10; or the following structure:

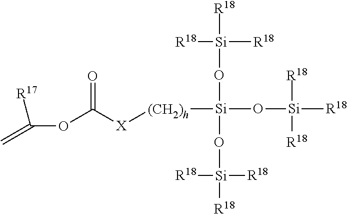

wherein X denotes —NR¹⁹—; wherein R¹⁹ denotes hydrogen or a C₁-C₄ alkyl; R¹⁷ denotes hydrogen or methyl; each R¹⁸ independently denotes a C₁-C₆ alkyl radical, a phenyl radical or a group represented by the following structure:

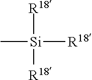

wherein each R^{18′} independently denotes a C₁-C₆ alkyl radical or a phenyl radical; and h is 1 to 10.

In non-limiting illustrative embodiments, as may be combined with one or more of the preceding paragraphs, the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group comprises a methacrylated lifitegrast or salt thereof-containing monomer or a methacrylated lifitegrast or salt thereof-containing polymer.

In non-limiting illustrative embodiments, as may be combined with one or more of the preceding paragraphs, the releasable linking group is attached to the lifitegrast or a salt thereof and the ethylenically unsaturated reactive group via a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the releasable linking group is a nitrobenzene group and the ethylenically unsaturated reactive group is a methacrylate group.

In non-limiting illustrative embodiments, as may be combined with one or more of the preceding paragraphs, the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group is the lifitegrast or salt thereof-containing polymer comprising an ethylenically unsaturated reactive group and a releasable linking group.

In non-limiting illustrative embodiments, as may be combined with one or more of the preceding paragraphs, the lifitegrast or salt thereof-containing polymer comprising an ethylenically unsaturated reactive group and a releasable linking group comprises hyaluronic acid comprising first repeating units comprising a methacrylate-containing residue and second repeating units comprising a lifitegrast or a salt thereof-containing residue.

In non-limiting illustrative embodiments, as may be combined with one or more of the preceding paragraphs, the monomer having a reactive functionality complementary to the hydroxyl moiety of the hydroxyalkyl acrylate monomer and a polymerizable ethylenically unsaturated reactive group is selected from the group consisting of 2-isocyanatoethyl acrylate, 3-isocyanatopropyl acrylate, 2-isocyanatoethyl methacrylate, 1-methyl-2-isocyanatoethyl methacrylate, 1,1-dimethyl-2-isocyanatoethyl acrylate, (meth)acryloyl chloride and vinyl chloroformate.

In non-limiting illustrative embodiments, as may be combined with one or more of the preceding paragraphs, the silicone prepolymer comprises:
from about 50 to about 400 repeating units of monomeric units derived from the alkylacrylamide monomer,
from about 50 to about 200 repeating units of monomeric units derived from the hydroxyalkyl acrylate monomer,
from about 5 to about 20 repeating units of monomeric units derived from the hydroxyalkyl acrylate monomer where the acrylate moiety is attached to the backbone of the silicone prepolymer and the hydroxy moiety is end functionalized with the monomer having a reactive functionality complementary to the hydroxyl moiety of the hydroxyalkyl acrylate and a polymerizable ethylenically unsaturated reactive group,
from about 20 to about 300 repeating units of monomeric units derived from the non-bulky organosilicon-containing monomer having an ethylenically unsaturated reactive group,
from about 1 to about 400 repeating units of monomeric units derived from the bulky siloxane monomer having an ethylenically unsaturated reactive group, and
from about 5 to about 50 repeating units of monomeric units derived from the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group.

In non-limiting illustrative embodiments, as may be combined with one or more of the preceding paragraphs, the polymerization composition further comprises (vi) an ultraviolet blocker having an ethylenically unsaturated reactive group.

In non-limiting illustrative embodiments, as may be combined with one or more of the preceding paragraphs, the silicone prepolymer further comprises from about 1 to about 25 repeating units of the monomeric units derived from an ultraviolet blocker having an ethylenically unsaturated reactive group.

In non-limiting illustrative embodiments, as may be combined with one or more of the preceding paragraphs, the polymerization composition further comprises a blue light blocker having an ethylenically unsaturated reactive group.

In non-limiting illustrative embodiments, as may be combined with one or more of the preceding paragraphs, the silicone prepolymer has a weight average molecular weight ranging from about 10,000 to about 300,000 Da.

According to another aspect of the present disclosure, a silicone contact lens comprising a polymerization product of a silicone contact lens-forming mixture comprising a silicone prepolymer comprising a reaction product of (a) a copolymerization product of a polymerization composition comprising (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group and (v) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (b) a monomer having a reactive functionality complementary to the hydroxyl moiety of the hydroxyalkyl acrylate monomer and a polymerizable ethylenically unsaturated reactive group.

In non-limiting illustrative embodiments, as may be combined with one or more of the preceding paragraphs, the alkylacrylamide monomer is N,N-dimethylacrylamide, and the hydroxyalkyl acrylate monomer is a hydroxyethyl acrylate monomer.

In non-limiting illustrative embodiments, as may be combined with one or more of the preceding paragraphs, the non-bulky organosilicon-containing monomer is represented by the following structure:

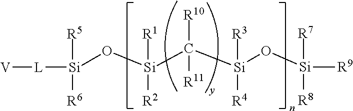

wherein V is an ethylenically unsaturated polymerizable group, L is a linking group or a bond; R¹, R², R³, R⁴, R⁵, R⁶, R⁷, R⁸, and R⁹ are independently hydrogen an alkyl group, a haloalkyl group, a cycloalkyl group, a heterocycloalkyl group, an alkenyl group, a halo alkenyl group, or an aromatic group; R¹⁰ and R¹¹ are independently hydrogen or alkyl, wherein at least one of R¹⁰ and R¹¹ is hydrogen; y is 2 to 7 and n is 1 to 100, or the non-bulky organosilicon-containing monomer is represented by the following structure:

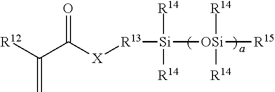

wherein R¹² is H or methyl; X is O or NR¹⁶, wherein R¹⁶ is hydrogen or C₁ to C₄ alkyl, which may be further substituted with one or more hydroxyl groups; R¹³ is a divalent alkyl group, which may further be functionalized with a group selected from the group consisting of an ether group, a hydroxyl group, a carbamate group and combinations thereof; each R¹⁴ is independently a phenyl or C₁ to C₄ alkyl which may be substituted with fluorine, hydroxyl or an ether; R¹⁵ is a C₁ to C₄ alkyl; and a is 2 to 50.

In non-limiting illustrative embodiments, as may be combined with one or more of the preceding paragraphs, the bulky siloxane monomer is selected from the group consisting of a bulky polysiloxanylalkyl (meth)acrylic monomer, a bulky polysiloxanylalkyl carbamate monomer and mixtures thereof.

In non-limiting illustrative embodiments, as may be combined with one or more of the preceding paragraphs, the bulky siloxane monomer is represented by the following structure:

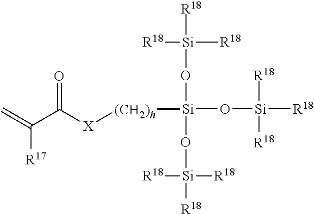

wherein X denotes —O— or —NR¹⁹— where each R¹⁹ is hydrogen or a C₁-C₄ alkyl; R¹⁷ independently denotes hydrogen or methyl; each R¹⁸ independently denotes a C₁-C₆ alkyl radical, a phenyl radical or a group represented by the following structure:

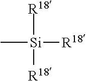

wherein each R^{18′} independently denotes a C₁-C₆ alkyl radical or a phenyl radical; and h is 1 to 10; or the following structure:

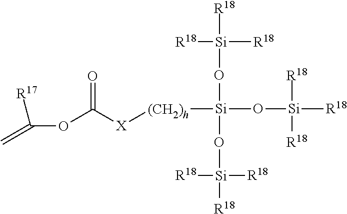

wherein X denotes —NR¹⁹—; wherein R¹⁹ denotes hydrogen or a C₁-C₄ alkyl; R¹⁷ denotes hydrogen or methyl; each R¹⁸ independently denotes a C₁-C₆ alkyl radical, a phenyl radical or a group represented by the following structure:

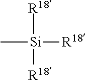

wherein each R^{18′} independently denotes a C₁-C₆ alkyl radical or a phenyl radical; and h is 1 to 10.

In non-limiting illustrative embodiments, as may be combined with one or more of the preceding paragraphs, the lifitegrast or salt thereof-containing monomer or polymer comprising ethylenically unsaturated reactive group and a photocleavable group comprises a methacrylated lifitegrast or salt thereof-containing monomer or polymer comprising the photocleavable group.

In non-limiting illustrative embodiments, as may be combined with one or more of the preceding paragraphs, the releasable linking group is attached to the lifitegrast or a salt thereof and the ethylenically unsaturated reactive group via a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the releasable linking group is a nitrobenzene group and the ethylenically unsaturated reactive group is a methacrylate group.

In non-limiting illustrative embodiments, as may be combined with one or more of the preceding paragraphs, the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group is the lifitegrast or salt thereof-containing polymer comprising an ethylenically unsaturated reactive group and a releasable linking group.

In non-limiting illustrative embodiments, as may be combined with one or more of the preceding paragraphs, the lifitegrast or salt thereof-containing polymer comprising an ethylenically unsaturated reactive group and a releasable linking group comprises hyaluronic acid comprising first repeating units comprising a methacrylate-containing residue and second repeating units comprising a lifitegrast or a salt thereof-containing residue.

In non-limiting illustrative embodiments, as may be combined with one or more of the preceding paragraphs, the monomer having a reactive functionality complementary to the hydroxyl moiety of the hydroxyalkyl acrylate monomer and a polymerizable ethylenically unsaturated reactive group is selected from the group consisting of 2-isocyanatoethyl acrylate, 3-isocyanatopropyl acrylate, 2-isocyanatoethyl methacrylate, 1-methyl-2-isocyanatoethyl methacrylate, 1,1-dimethyl-2-isocyanatoethyl acrylate, (meth)acryloyl chloride and vinyl chloroformate.

In non-limiting illustrative embodiments, as may be combined with one or more of the preceding paragraphs, the silicone prepolymer comprises:
from about 50 to about 400 repeating units of monomeric units derived from the alkylacrylamide monomer,
from about 50 to about 200 repeating units of monomeric units derived from the hydroxyalkyl acrylate monomer,
from about 5 to about 20 repeating units of monomeric units derived from the hydroxyalkyl acrylate monomer where the acrylate moiety is attached to the backbone of the silicone prepolymer and the hydroxy moiety is end functionalized with the monomer having a reactive functionality complementary to the hydroxyl moiety of the hydroxyalkyl acrylate and a polymerizable ethylenically unsaturated reactive group,
from about 20 to about 300 repeating units of monomeric units derived from the non-bulky organosilicon-containing monomer having an ethylenically unsaturated reactive group,
from about 1 to about 400 repeating units of the monomeric units derived from the bulky siloxane monomer having an ethylenically unsaturated reactive group, and
from about 5 to about 50 repeating units of monomeric units derived from the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group.

In non-limiting illustrative embodiments, as may be combined with one or more of the preceding paragraphs, the polymerization composition further comprises (v) an ultraviolet blocker having an ethylenically unsaturated reactive group.

In non-limiting illustrative embodiments, as may be combined with one or more of the preceding paragraphs, the silicone prepolymer further comprises from about 1 to about 25 repeating units of monomeric units derived from an ultraviolet blocker having an ethylenically unsaturated reactive group.

In non-limiting illustrative embodiments, as may be combined with one or more of the preceding paragraphs, the polymerization composition further comprises a blue light blocker having an ethylenically unsaturated reactive group.

In non-limiting illustrative embodiments, as may be combined with one or more of the preceding paragraphs, the silicone prepolymer has a weight average molecular weight ranging from about 10,000 to about 300,000 Da.

In non-limiting illustrative embodiments, as may be combined with one or more of the preceding paragraphs, the silicone prepolymer is present in the silicone contact lens-forming mixture in an amount ranging from about 50 wt. % to about 100 wt. %, based on the total weight of the silicone contact lens-forming mixture.

In non-limiting illustrative embodiments, as may be combined with one or more of the preceding paragraphs, the silicone prepolymer is present in the silicone contact lens-forming mixture in an amount ranging from about 60 wt. % to about 80 wt. %, based on the total weight of the silicone contact lens-forming mixture.

In non-limiting illustrative embodiments, as may be combined with one or more of the preceding paragraphs, the silicone contact lens-forming mixture further comprises one or more silicone contact lens-forming monomers.

In non-limiting illustrative embodiments, as may be combined with one or more of the preceding paragraphs, the one or more silicone contact lens-forming monomers are present in the silicone contact lens-forming mixture in an amount ranging from about 10 wt. % to about 50 wt. %, based on the total weight of the silicone contact lens-forming mixture.

In non-limiting illustrative embodiments, as may be combined with one or more of the preceding paragraphs, the silicone contact lens-forming mixture further comprises one or more hydrophilic comonomers.

In non-limiting illustrative embodiments, as may be combined with one or more of the preceding paragraphs, the one or more hydrophilic comonomers include one or more of an unsaturated carboxylic acid, an acrylamide, a vinyl lactam, a hydroxyl-containing-(meth)acrylate, a hydrophilic vinyl carbonate, a hydrophilic vinyl carbamate, a hydrophilic oxazolone, and a poly(alkene glycols) functionalized with polymerizable groups and mixtures thereof.

In non-limiting illustrative embodiments, as may be combined with one or more of the preceding paragraphs, the one or more hydrophilic comonomers are present in the silicone contact lens-forming mixture in an amount ranging from about 10 wt. % to about 50 wt. %, based on the total weight of the silicone contact lens-forming mixture.

In non-limiting illustrative embodiments, as may be combined with one or more of the preceding paragraphs, the silicone contact lens-forming mixture further comprises one or more blue light blockers having an ethylenically unsaturated reactive group.

In non-limiting illustrative embodiments, as may be combined with one or more of the preceding paragraphs, the one or more blue light blockers are represented by an acridone compound represented by the following structure:

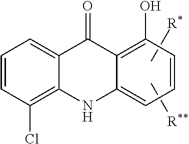

wherein R* is hydrogen or a substituted or unsubstituted hydrocarbyl group, and R** is an ethylenically unsaturated reactive group.

In non-limiting illustrative embodiments, as may be combined with one or more of the preceding paragraphs, the silicone contact lens has an oxygen permeability of at least about 50 Barrers.

In non-limiting illustrative embodiments, as may be combined with one or more of the preceding paragraphs, the silicone contact lens has an oxygen permeability of from about 75 Barrers to about 175 Barrers.

In non-limiting illustrative embodiments, as may be combined with one or more of the preceding paragraphs, the lifitegrast or salt thereof is released in a sustained release manner.

According to another aspect of the present disclosure, a method for making a silicone contact lens comprises:
(a) subjecting a silicone contact lens-forming mixture comprising a silicone prepolymer of the present disclosure to polymerization conditions to provide a polymerized silicone contact lens, and
(b) hydrating the polymerized silicone contact lens.

In non-limiting illustrative embodiments, as may be combined with one or more of the preceding paragraphs, the silicone prepolymer is present in the contact lens-forming mixture in an amount ranging from about 50 wt. % to about 100 wt. %, based on the total weight of the silicone contact lens-forming mixture.

In non-limiting illustrative embodiments, as may be combined with one or more of the preceding paragraphs, the silicone prepolymer is present in the silicone contact lens-forming mixture in an amount ranging from about 60 wt. % to about 80 wt. %, based on the total weight of the silicone contact lens-forming mixture.

In non-limiting illustrative embodiments, as may be combined with one or more of the preceding paragraphs, the silicone contact lens-forming mixture further comprises one or more silicone contact lens-forming monomers.

In non-limiting illustrative embodiments, as may be combined with one or more of the preceding paragraphs, the one or more silicone contact lens-forming monomers are present in the silicone contact lens-forming mixture in an amount ranging from about 10 wt. % to about 50 wt. %, based on the total weight of the silicone contact lens-forming mixture.

In non-limiting illustrative embodiments, as may be combined with one or more of the preceding paragraphs, the silicone contact lens-forming mixture further comprises one or more hydrophilic comonomers.

In non-limiting illustrative embodiments, as may be combined with one or more of the preceding paragraphs, the one or more hydrophilic comonomers include one or more of an unsaturated carboxylic acid, an acrylamide, a vinyl lactam, a hydroxyl-containing-(meth)acrylate, a hydrophilic vinyl carbonate, a hydrophilic vinyl carbamate, a hydrophilic oxazolone, and a poly(alkene glycols) functionalized with polymerizable groups and mixtures thereof.

In non-limiting illustrative embodiments, as may be combined with one or more of the preceding paragraphs, the one or more hydrophilic comonomers are present in the silicone contact lens-forming mixture in an amount ranging from about 10 wt. % to about 50 wt. %, based on the total weight of the silicone contact lens-forming mixture.

In non-limiting illustrative embodiments, as may be combined with one or more of the preceding paragraphs, the silicone contact lens has an oxygen permeability of at least about 50 Barrers.

In non-limiting illustrative embodiments, as may be combined with one or more of the preceding paragraphs, the silicone contact lens has an oxygen permeability of from about 75 Barrers to about 175 Barrers.

According to another aspect of the present disclosure, a method for delivering lifitegrast or salt thereof to an eye of a subject comprises:
(a) placing a silicone contact lens in an eye of a subject, wherein the silicone contact lens is a polymerization product of a silicone contact lens-forming mixture comprising a silicone prepolymer of the present disclosure, and
(b) delivering the lifitegrast or salt thereof to the eye of the subject by contacting the silicone contact lens with one of light having a wavelength of about 300 nanometers (nm) to about 700 nm or with an esterases or enzymes on the eye, in the tear film or on the flora of the eye.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

1. A silicone prepolymer comprising a reaction product of (a) a copolymerization product of a polymerization composition comprising (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group and (v) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (b) a monomer having a reactive functionality complementary to the hydroxyl moiety of the hydroxyalkyl acrylate monomer and a polymerizable ethylenically unsaturated reactive group.

2. The silicone prepolymer according to claim 1, wherein the alkylacrylamide monomer is N,N-dimethylacrylamide, and the hydroxyalkyl acrylate monomer is a hydroxyethyl acrylate monomer.

3. The silicone prepolymer according to claim 1, wherein the non-bulky organosilicon-containing monomer is represented by the following structure: 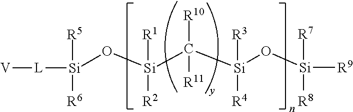
wherein V is an ethylenically unsaturated polymerizable group, L is a linking group or a bond; R¹, R², R³, R⁴, R⁵, R⁶, R⁷, R⁸, and R⁹ are independently hydrogen an alkyl group, a haloalkyl group, a cycloalkyl group, a heterocycloalkyl group, an alkenyl group, a halo alkenyl group, or an aromatic group; R¹⁰ and R¹¹ are independently hydrogen or alkyl, wherein at least one of R¹⁰ and R¹¹ is hydrogen; y is 2 to 7 and n is 1 to 100, or the non-bulky organosilicon-containing monomer is represented by the following structure: 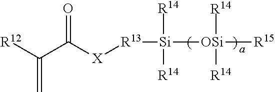
wherein R¹² is H or methyl; X is O or NR¹⁶, wherein R¹⁶ is hydrogen or C₁ to C₄ alkyl, which may be further substituted with one or more hydroxyl groups; R¹³ is a divalent alkyl group, which may further be functionalized with a group selected from the group consisting of an ether group, a hydroxyl group, a carbamate group and combinations thereof; each R¹⁴ is independently a phenyl or C₁ to C₄ alkyl which may be substituted with fluorine, hydroxyl or an ether; R¹⁵ is a C₁ to C₄ alkyl; and a is 2 to 50.

4. The silicone prepolymer according to claim 1, wherein the bulky siloxane monomer is represented by the following structure: 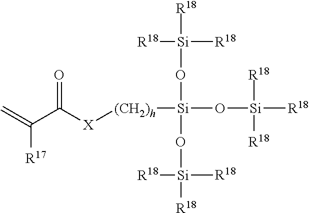
wherein X denotes —O— or —NR¹⁹— where each R¹⁹ is hydrogen or a C₁-C₄ alkyl; R¹⁷ independently denotes hydrogen or methyl; each R¹⁸ independently denotes a C₁-C₆ alkyl radical, a phenyl radical or a group represented by the following structure: 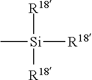
wherein each R^{18′} independently denotes a C₁-C₆ alkyl radical or a phenyl radical; and h is 1 to 10; or the following structure: 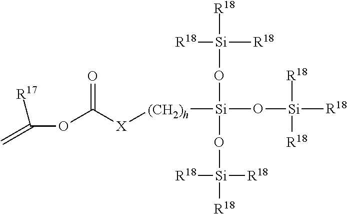
wherein X denotes —NR¹⁹—; wherein R¹⁹ denotes hydrogen or a C₁-C₄ alkyl; R¹⁷ denotes hydrogen or methyl; each R¹⁸ independently denotes a C₁-C₆ alkyl radical, a phenyl radical or a group represented by the following structure: 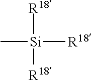
wherein each R^{18′} independently denotes a C₁-C₆ alkyl radical or a phenyl radical; and his 1 to 10.

5. The silicone prepolymer according to claim 1, wherein the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group comprises a methacrylated lifitegrast or salt thereof-containing monomer or a methacrylated lifitegrast or salt thereof-containing polymer.

6. The silicone prepolymer according to claim 1, wherein the releasable linking group is attached to the lifitegrast or a salt thereof and the ethylenically unsaturated reactive group via a pH-labile bond, an acid-labile bond, a base-labile bond, an oxidatively labile bond, a metabolically labile bond, a biochemically labile bond, an enzyme-labile bond, a chemical-labile bond or a photo-labile bond.

7. The silicone prepolymer according to claim 1, wherein the releasable linking group is a nitrobenzene group and the ethylenically unsaturated reactive group is a methacrylate group.

8. The silicone prepolymer according to claim 1, wherein the lifitegrast or salt thereof-containing polymer comprising an ethylenically unsaturated reactive group and a releasable linking group comprises hyaluronic acid comprising first repeating units comprising a methacrylate-containing residue and second repeating units comprising a lifitegrast or salt thereof-containing residue.

9. The silicone prepolymer according to claim 1, wherein the monomer having a reactive functionality complementary to the hydroxyl moiety of the hydroxyalkyl acrylate monomer and a polymerizable ethylenically unsaturated reactive group is selected from the group consisting of 2-isocyanatoethyl acrylate, 3-isocyanatopropyl acrylate, 2-isocyanatoethyl methacrylate, 1-methyl-2-isocyanatoethyl methacrylate, 1,1-dimethyl-2-isocyanatoethyl acrylate, (meth)acryloyl chloride and vinyl chloroformate.

10. The silicone prepolymer according to claim 1, wherein the silicone prepolymer comprises:
from about 50 to about 400 repeating units of monomeric units derived from the alkylacrylamide monomer;
from about 50 to about 200 repeating units of monomeric units derived from the hydroxyalkyl acrylate monomer;
from about 5 to about 20 repeating units of monomeric units derived from the hydroxyalkyl acrylate monomer where the acrylate moiety is attached to the backbone of the silicone prepolymer and the hydroxy moiety is end functionalized with the monomer having a reactive functionality complementary to the hydroxyl moiety of the hydroxyalkyl acrylate and a polymerizable ethylenically unsaturated reactive group;
from about 20 to about 300 repeating units of monomeric units derived from the non-bulky organosilicon-containing monomer having an ethylenically unsaturated reactive group;
from about 1 to about 400 repeating units of monomeric units derived from the bulky siloxane monomer having an ethylenically unsaturated reactive group; and
from about 5 to about 50 repeating units of monomeric units derived from the one of the lifitegrast or salt thereof-containing monomer comprising an ethylenically unsaturated reactive group and a releasable linking group or the lifitegrast or salt thereof-containing polymer comprising an ethylenically unsaturated reactive group and a releasable linking group.

11. The silicone prepolymer according to claim 10, wherein the silicone prepolymer further comprises from about 1 to about 25 repeating units of monomeric units derived from an ultraviolet blocker having an ethylenically unsaturated reactive group.

12. A silicone contact lens comprising a polymerization product of a silicone contact lens-forming mixture comprising a silicone prepolymer comprising a reaction product of (a) a copolymerization product of a polymerization composition comprising (i) an alkylacrylamide monomer, (ii) a hydroxyalkyl acrylate monomer, (iii) a non-bulky organosilicon-containing monomer having an ethylenically unsaturated reactive group, (iv) a bulky siloxane monomer having an ethylenically unsaturated reactive group and (v) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (b) a monomer having a reactive functionality complementary to the hydroxyl moiety of the hydroxyalkyl acrylate monomer and a polymerizable ethylenically unsaturated reactive group.

13. The silicone contact lens according to claim 12, wherein the releasable linking group is an ester group and the ethylenically unsaturated reactive group is a methacrylate group.

14. The silicone contact lens according to claim 12, wherein the releasable linking group is a nitrobenzene group and the ethylenically unsaturated reactive group is a methacrylate group.

15. The silicone contact lens according to claim 12, wherein the lifitegrast or salt thereof-containing polymer comprising an ethylenically unsaturated reactive group and a releasable linking group comprises hyaluronic acid comprising first repeating units comprising a methacrylate-containing residue and second repeating units comprising a lifitegrast or a salt thereof-containing residue.

16. The silicone contact lens according to claim 12, wherein the silicone prepolymer comprises:
from about 50 to about 400 repeating units of monomeric units derived from the alkylacrylamide monomer;
from about 50 to about 200 repeating units of monomeric units derived from the hydroxyalkyl acrylate monomer;
from about 5 to about 20 repeating units of monomeric units derived from the hydroxyalkyl acrylate monomer where the acrylate moiety is attached to the backbone of the silicone prepolymer and the hydroxy moiety is end functionalized with the monomer having a reactive functionality complementary to the hydroxyl moiety of the hydroxyalkyl acrylate and a polymerizable ethylenically unsaturated reactive group;
from about 20 to about 300 repeating units of monomeric units derived from the non-bulky organosilicon-containing monomer having an ethylenically unsaturated reactive group;
from about 1 to about 400 repeating units of monomeric units derived from the bulky siloxane monomer having an ethylenically unsaturated reactive group; and
from about 5 to about 50 repeating units of monomeric units derived from the one of the lifitegrast salt thereof-containing monomer comprising an ethylenically unsaturated reactive group and a releasable linking group or the lifitegrast or salt thereof-containing polymer comprising an ethylenically unsaturated reactive group and a releasable linking group.

17. The silicone contact lens according to claim 16, wherein the silicone prepolymer further comprises from about 1 to about 25 repeating units of monomeric units derived from an ultraviolet blocker having an ethylenically unsaturated reactive group.

18. The silicone contact lens according to claim 12, wherein the silicone contact lens-forming mixture further comprises one or more silicone contact lens-forming monomers, one or more hydrophilic comonomers, or both.

19. The silicone contact lens according to claim 12, wherein the lifitegrast or salt thereof is released in a sustained release manner.

20. A method for making a silicone contact lens, comprising:
(a) subjecting a silicone contact lens-forming mixture comprising a silicone prepolymer comprising a reaction product of (i) a copolymerization product of a polymerization composition comprising (1) an alkylacrylamide monomer, (2) a hydroxyalkyl acrylate monomer, (3) a non-bulky organosilicon-containing monomer having an ethylenically unsaturated reactive group, (4) a bulky siloxane monomer having an ethylenically unsaturated reactive group, and (5) one of a lifitegrast or a salt thereof-containing monomer comprising an ethylenically unsaturated reactive group and a releasable linking group or a lifitegrast or a salt thereof-containing polymer comprising an ethylenically unsaturated reactive group and a releasable linking group, with (ii) a monomer having a reactive functionality complementary to the hydroxyl moiety of the hydroxyalkyl acrylate monomer and a polymerizable ethylenically unsaturated reactive group to polymerization conditions to provide a polymerized silicone contact lens; and
(b) hydrating the polymerized silicone contact lens.

21. The silicone contact lens according to claim 12, wherein the silicone prepolymer is present in the silicone contact lens-forming mixture in an amount ranging from about 50 wt. % to about 100 wt. %, based on the total weight of the silicone contact lens-forming mixture.

22. A method for delivering lifitegrast or a salt thereof to an eye of a subject, comprising:
(a) placing the silicone contact lens according to claim 12 in an eye of a subject; and
(b) delivering the lifitegrast or salt thereof to the eye of the subject by contacting the silicone contact lens with one of light having a wavelength of about 300 nanometers (nm) to about 700 nm or with an esterases or enzymes on the eye, in a tear film or on a flora of the eye.
